(19) United States
(12) Patent Application Publication
(10) Pub. No.: US 2026/0294821 A1
(43) Pub. Date: Oct. 1, 2026

(54) MAGNETICALLY DRIVENABLE ANTIGEN DELIVERY UNIT AND METHOD OF FORMING SAME

(71) Applicant: CITY UNIVERSITY OF HONG KONG, Kowloon (HK)

(72) Inventors: Dong SUN, Kowloon (HK); Qi ZHANG, Kowloon (HK); Junyang LI, Shenzhen (CN)

(21) Appl. No.: 19/089,057

(22) Filed: Mar. 25, 2025

(51) Int. Cl. A61K 9/50; A61K 9/51; A61K 39/00; B82Y 5/00

(52) U.S. Cl. CPC A61K 9/5057; A61K 9/5115; A61K 39/0005; B82Y 5/00

(57) ABSTRACT

A magnetically drivenable antigen delivery unit is disclosed as including a magnetic nanoparticle to which an antigen is loaded, and a biodegradable material encapsulating the magnetic nanoparticle. A method of forming a magnetically drivenable antigen delivery unit using microfluidic droplet generation technique is disclosed as including forming a solution of a magnetic nanoparticle to which an antigen is loaded, gelatin methacryloyl (GelMA), and lithium phenyl-2,4,6-trimethylbenzoylphosphinate (LAP). The invention also discloses a method for realizing lymph node targeted delivery by magnetically driving subunit antigens.

### TECHNICAL FIELD OF THE INVENTION

The invention relates, generally, to the field of magnetically drivenable antigen (subunit vaccine) delivery unit, a method of forming such a unit, and a method of delivering an antigen (subunit vaccine) to a subject, e.g., to or adjacent to a targeted location of a subject.

### BACKGROUND OF THE INVENTION

Vaccination is a cornerstone in controlling infectious diseases, thus significantly reducing mortality rates. Both traditional vaccines, derived from attenuated or inactivated pathogens, and modern DNA or mRNA-based vaccines have demonstrated efficacy against diverse infectious diseases such as the new coronavirus that began to circulate in 2019. Nevertheless, such potential adverse effects as inflammation, allergies, and autoimmune reactions of vaccines restrict their widespread adoption. Subunit vaccines, comprising protein as antigenic components, offer distinct advantages over other vaccine types due to their enhanced safety profile, cost-effective production methods, and logistical benefits, including temperature stability. Effective vaccine delivery to lymph nodes is pivotal for optimizing vaccine availability and retention time.

Lymph nodes are rich in antigen-presenting cells (APCs), including dendritic cells (DCs), and the leading site of immune activation. Protein vaccines must undergo an efficient in vivo cascade process to generate an effective cell-mediated immune response and maximize overall immune efficacy. Recent studies have shown that 10-100 nm nanovesicles can be effectively drained through lymphatic endothelial cell gaps to lymph node locations and promote DCs activation and antigen cross-presentation, thereby eliciting robust cellular adaptive immunity. In addition, previous studies have shown that vesicles with negative, hydrophilic surfaces can be efficiently drained to lymph nodes, thereby improving antigen cross-presentation capacity and inducing an effective immune response. After antigen-laden nanoparticles drain to the lymph nodes, they are internalized by DCs; subsequently, the activation of these dendritic cells and antigen cross-presentation play a pivotal role in triggering robust adaptive immunity. Magnetic nanoparticles (MNPs) demonstrate the advantages of easy surface modification, tunable particle size, and biocompatibility, making them extensively investigated for vaccine delivery.

Drug-loaded nanoparticles face challenges in achieving sustained release because they are susceptible to drug loss during delivery. Various magnetic microstructures have been developed to precisely deliver therapeutic agents to targeted locations to address this challenge. The drug-loading function in microstructures designed for delivering drug-loaded MNPs is typically achieved through chemical interactions between the nanoparticles and the surface of the microstructure. However, the fabrication of most of these mobile drug-loaded microstructures relies on two-photon polymerization (TPP) technology, a method that is both time-consuming and low-throughput. Furthermore, high concentrations of magnetic particles significantly limit the manufacturing efficiency and quality of TPP.

Droplet microfluidics technology has gained significant attention among drug delivery researchers due to its high-throughput characteristics and wide application in pharmaceuticals and material synthesis. Previous studies have demonstrated that these droplet-generation structures can achieve thousands-per-second production rates, highlighting the high-throughput nature of droplet microfluidics. Contemporary droplet microfluidic devices primarily employ flow-focusing, T-junction, and coflow configurations as the primary structures for droplet generation. These droplets are critical for subsequent material synthesis, chemical reactions, and targeted drug delivery in the microfluidic process. Among them, gelatin methacryloyl (GelMA) droplets, due to the selection of an enzyme-degradable material, possess excellent cytotoxicity and photopolymerization characteristics, as well as the ability to effectively protect encapsulated drugs, making them widely used in the generation of microgels using droplet microfluidic chips.

Despite the above advances, most existing subunit nanovaccines lack the functionality of antigen-targeted delivery. In addition, many existing drug microcarriers do not allow for sustained release of vaccines.

It is thus an objective of the present invention to provide a magnetically drivenable antigen delivery unit, a method of forming a magnetically drivenable antigen delivery unit, and a method of delivering an antigen to a subject in which at least one of the above shortcomings is mitigated, or at least to provide a useful alternative to the trade and public.

### SUMMARY OF THE INVENTION

According to a first aspect of the present invention, there is provided a magnetically drivenable antigen delivery unit, including a magnetic nanoparticle to which a subunit antigen is loaded, and a biodegradable material encapsulating said magnetic nanoparticle.

According to a second aspect of the present invention, there is provided a method of forming a magnetically drivenable antigen delivery unit, including a step (a) of forming a first solution including at least one magnetic nanoparticle to which a subunit antigen is loaded, gelatin methacryloyl (GelMA), and lithium phenyl-2,4,6-trimethylbenzoylphosphinate (LAP).

According to a third aspect of the present invention, there is provided a method of delivering an antigen to a subject, including injecting into said subject at least one magnetically drivenable antigen delivery unit including a magnetic nanoparticle to which a subunit antigen is loaded, and a biodegradable material encapsulating said magnetic nanoparticle.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of examples only, with reference to the accompanying drawings, in which:

FIG. 1A is a schematic diagram of a process according to an embodiment of the present invention for the formation of ovalbumin@magnetic nanoparticles (OVA@MNPs) loaded (OVA@MNPs-loaded) GelMA microspheres (OMGMs) using microfluidic droplet generation technology;

FIG. 1B is a flow chart showing steps for fabricating OVA@MNPs and OMGMs according to an embodiment of the present invention;

FIG. 2 is a schematic diagram of the use of OMGMs for subunit vaccine (antigen) delivery and enhanced immunotherapy;

FIG. 3 is a schematic diagram of a process according to an embodiment of the present invention for the synthesis of OVA@MNPs and the preparation of GelMA-based precursor;

FIG. 4 shows distribution of nanoparticle size before and after synthesis of OVA@MNPs according to the present invention;

FIG. 5 shows size comparison of MNPs and OVA@MNPs according to the present invention;

FIG. 6 shows Zeta potential comparison of MNPs and OVA@MNPs according to the present invention;

FIG. 7 shows transmission electron microscopy (TEM) image of OVA@MNPs according to the present invention; scale bar of the enlarged image, 50 nm; scale bar of the original image, 100 nm;

FIG. 8 shows ovalbumin (OVA) loading efficiency and loading capacity;

FIG. 9 shows cumulative release percentage of OVA from OVA@MNPs according to the present invention in phosphate-buffered saline (PBS);

FIG. 10 is a schematic view showing the fabrication of OMGMs according to the present invention by using a microfluidic chip, in which the enlarged part shows the focusing junction details of generating the OMGMs according to the present invention, including inset (I) showing the simulation result and insets (II) showing the optical microscope image;

FIG. 11 shows schematic steps of fabricating a microfluidic chip for generating OMGMs according to the present invention;

FIG. 12 shows a microfluidic chip fabricated for generating OMGMs according to the present invention;

FIG. 13 shows the optical microscope image of the OMGMs according to the present invention in Dulbecco's modified eagle medium (DMEM); scale bar, 20 μm;

FIG. 14 shows magnetic response of the OMGMs according to the present invention in a tube;

FIG. 15 shows X-ray diffractometer (XRD) results of the GelMA-based precursor and OMGMs according to the present invention;

FIG. 16 shows a scanning electron microscope (SEM) image of the OMGMs according to the present invention; scale bar, 20 μm;

FIG. 17 shows energy-dispersive X-ray spectroscopy (EDS) elemental mapping of the OMGMs according to the present invention;

FIG. 18 shows a SEM result of OMGMs chart with EDS elemental analysis; scale bar, 20 μm;

FIG. 19 shows DC2.4 cells viability using varying concentrations of MNPs for 24 and 72 h;

FIG. 20 shows culture status of DC2.4 cells exposed to OMGMs according to the present invention for 0 h, 12 h, and 24 h in which the dotted circles represent OMGMs; scale bar, 100 μm;

FIG. 21 shows mean fluorescence intensity of OMGMs according to the present invention when degraded in different concentrations of trypsin-EDTA (TE);

FIG. 22 shows fluorescence images of OMGMs according to the present invention degraded in 2.5 μg mL-1 TE for 50 min;

FIG. 23 shows DC2.4 cells viability using varying concentrations of the degradation products of OMGMs according to the present invention for 24 and 72 h;

FIG. 24 shows total radiant efficiency change curve of OVA@MNPs and OMGMs according to the present invention in mice for 72 h;

FIG. 25 shows in vivo biodegradation of OMGMs according to the present invention for 72 h, as observed Alexa fluor 647-OVA@MNPs using an IVIS instrument;

FIG. 26 shows in vivo biodegradation of OMGMs according to the present invention for 72 h, as observed GelMA-FITC using an IVIS instrument;

FIG. 27 shows magnetization of the OMGMs according to the present invention;

FIG. 28 shows velocities of the OMGMs according to the present invention under a gradient magnetic field;

FIG. 29 shows a schematic view of a system for generating a rotating magnetic field;

FIG. 30 shows a schematic view of a rolling motion control system;

FIG. 31 shows velocities of the OMGMs according to the present invention under a rotating magnetic field;

FIG. 32 shows magnetic actuation of the OMGMs according to the present invention traversing a rectangular path under a (4 Hz, 30 mT) magnetic field; scale bar, 200 μm.

FIGS. 33A and 33B show magnetic actuation of the OMGMs according to the present invention in the DC2.4 cell culture environment with RPMI-1640 medium under a (3 Hz, 30 mT) magnetic field; scale bar, 200 μm;

FIG. 34 shows uptake qualitative results using OVA@MNPs according to the present invention to culture with DC2.4 cells for 2-6 h; scale bar, 100 μm;

FIG. 35 shows uptake qualitative results after co-culture of macrophages and Alexa Fluor 647-OVA@MNPs for 2-6 h; scale bar, 50 μm;

FIG. 36 shows uptake comparison results using free OVA-FITC and OVA-FITC@MNPs loading with the same amount of OVA to culture with DC2.4 cells for 3 h; scale bar, 200 μm;

FIG. 37 shows relative uptake efficiency of OVA@MNPs according to the present invention compared with an equivalent amount of free OVA;

FIG. 38 shows mean fluorescence intensity of DC2.4 cells and RAW264.7 macrophages after 2-6 h of culturing with OVA@MNPs according to the present invention;

FIG. 39 shows exploration of internalization mechanisms of OVA@MNPs uptaken by DC2.4 cells;

FIG. 40 shows confocal laser scanning micrographs of exploring colocalization of DC2.4 cells exposed to OVA-Alexa Fluor 647@MNPs for 1 h, then cultured in the absence of drug for the indicated times. Green signal, to lysosomes stained with LysoTracker Green; red signal corresponds to OVA; scale bar, 5 μm;

FIG. 41 shows cross-presentation performance of OVA in DC2.4 cells;

FIG. 42 shows CD80 costimulatory molecule expression in bone marrow derived dendritic cells (BMDCs) following 18 h of incubation;

FIG. 43 shows CD86 costimulatory molecule expression in BMDCs following 18 h of incubation;

FIG. 44 shows induced immune response effects on DC2.4 cells by measuring TNF-α;

FIG. 45 shows induced immune response effects on DC2.4 cells by measuring IL-6;

FIG. 46 shows induced immune response effects on DC2.4 cells by measuring IFN-γ;

FIG. 47 shows induced immune response effects on macrophages by measuring TNF-α;

FIG. 48 shows induced immune response effects on macrophages by measuring IL-6;

FIG. 49 shows induced immune response effects on macrophages by measuring IFN-γ;

FIG. 50 shows injection setup to verify in vivo draining and targeting performance;

FIG. 51 is a schematic view of the distribution of sciatic lymph nodes and popliteal lymph nodes in mice;

FIG. 52 shows in vivo migration of Alexa Fluor 647-OVA from the injection site to the lymph nodes using an IVIS instrument;

FIG. 53 shows radiant efficiency of sciatic lymph nodes (SLN) within 36 h;

FIG. 54 shows radiant efficiency of popliteal lymph nodes (PLN) within 36 h;

FIG. 55 shows images of separated popliteal lymph nodes (PLN) and inguinal lymph nodes (ILN) after 36 h;

FIG. 56 shows quantification of radiant efficiency in isolated popliteal lymph nodes at 36 h;

FIG. 57 shows radiant efficiency of isolated inguinal lymph nodes after 36 h;

FIG. 58 is a schematic view of vaccination and blood sampling design;

FIG. 59 shows quantification of serum anti-OVA IgG antibody secretion levels using ELISA;

FIG. 60 shows secretion levels of IFN-α treated with saline, OVA, OVA@MNPs according to the present invention, and OMGMs according to the present invention on day 35;

FIG. 61 shows secretion levels of TNF-γ treated with saline, OVA, OVA@MNPs according to the present invention, and OMGMs according to the present invention on day 35;

FIG. 62 is a schematic view of a tumor challenge experimental design;

FIG. 63 shows tumor growth curves after immunization with OVA, OVA@MNPs according to the present invention, and OMGMs according to the present invention;

FIG. 64 shows tumor growth curves immunized with saline, OVA, OVA@MNPs, and OMGMs;

FIG. 65 shows tumor images after immunization with saline, OVA, OVA@MNPs according to the present invention, and OMGMs according to the present invention after mice sacrifice; scale bar, 1 cm;

FIG. 66 shows flow cytometer analysis of CD4+ IFN-γ+ T cells in spleens;

FIG. 67 shows flow cytometer analysis of CD8a+ IFN-γ+ T cells in the spleens;

FIG. 68 shows flow cytometry quantitative results of CD4+ IFN-γ+ T cells in the spleens of mice after various treatments;

FIG. 69 shows flow cytometer analysis of CD8a+ IFN-γ+ T cells in spleens after various treatments; and

FIG. 70 shows images of tissues for exploring biosafety of using a magnetically drivenable subunit vaccine (antigen) delivery unit, a method of forming such a unit, and a method of delivering an antigen to a subject of the present invention.

### DETAILED DESCRIPTION OF THE EMBODIMENTS

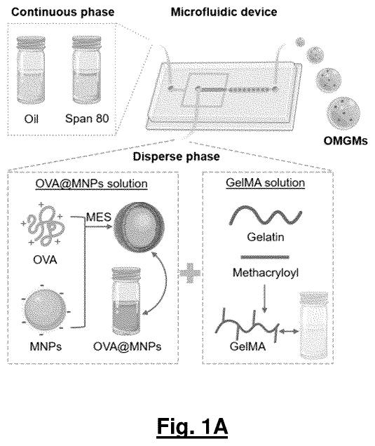

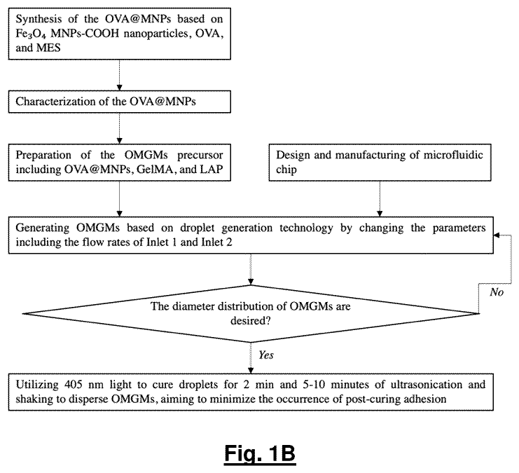

Broadly stated, the present invention relates to a novel subunit vaccine delivery unit utilizing ovalbumin@magnetic nanoparticles (OVA@MNPs) encapsulated within biodegradable gelatin methacryloyl (GelMA) microspheres to enhance the efficacy of antigen delivery, as shown in FIG. 1A. OVA@MNPs subunit vaccines (antigens) were synthesized, smaller than 100 nanometers and possessing a negatively charged surface. These OVA@MNPs efficiently deliver antigens intracellularly and facilitate antigen presentation. The synthesized OVA@MNPs were encapsulated using a biodegradable GelMA material to protect the subunit vaccines from rapid clearance. A microfluidic chip was employed to achieve high-throughput generation of magnetically driven OVA@MNPs-loaded GelMA microspheres (OMGMs), which exhibit minimal cytotoxicity and robust magnetic responsiveness.

As shown in FIG. 1B, according to an embodiment of the present invention, OVA@MNPs were synthesized based on Fe₃O₄ MNPs-COOH nanoparticles, OVA and 2-(N-morpholino) ethanesulfonic acid (MES). The precursors for the preparation of OMGMs include OVA@MNPs, GelMA and LAP. OMGMs in the form of microspheres were generated based on droplet generation technology, by changing the parameters, such as the flow rates of Inlet 1 and Inlet 2. During generation of the OMGMs, the diameter distribution of OMGMs was monitored to see if such is within the desired range. The parameters were adjusted when the diameter distribution of OMGMs was found to be beyond the desired range. OMGM microspheres were cured by 405 nm light for 2 min, subjected to ultrasonication for 5-10 min, and shaken to disperse the OMGMs, aiming to minimize post-curing adhesion of the OMGMs.

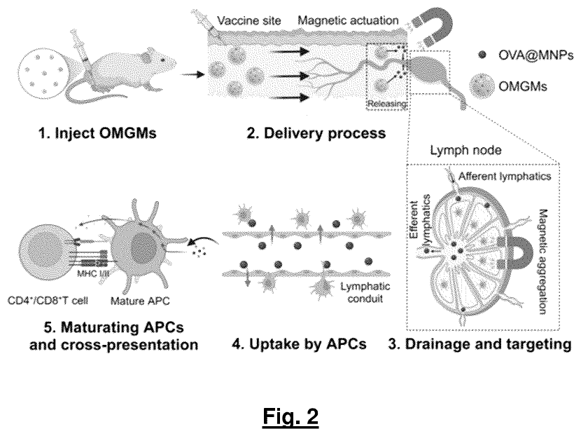

Due to the sustained degradation properties of OMGMs, the encapsulated OVA@MNPs are continuously released. Upon in vivo injection of the OMGMs into the body of a subject, their magnetic properties facilitate targeted delivery of antigen to the vicinity of lymph nodes within the body of the subject with the assistance of an external magnetic field. Additionally, the nano-size and surface charge of OVA@MNPs ensure adequate drainage to lymph nodes, thereby delivering more subunit vaccines to the lymph nodes rich in APCs. Subsequently, OVA@MNPs are efficiently endocytosed by APCs, leading to robust antigen presentation. Mature APCs can induce specific CD8+ T cells and CD4+ T cells, enhancing cellular and humoral immune responses (see FIG. 2).

### Synthesis and Characterization of OVA@MNPs and OMGMs

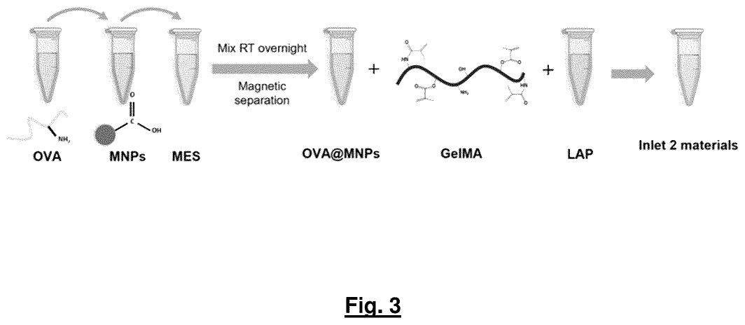

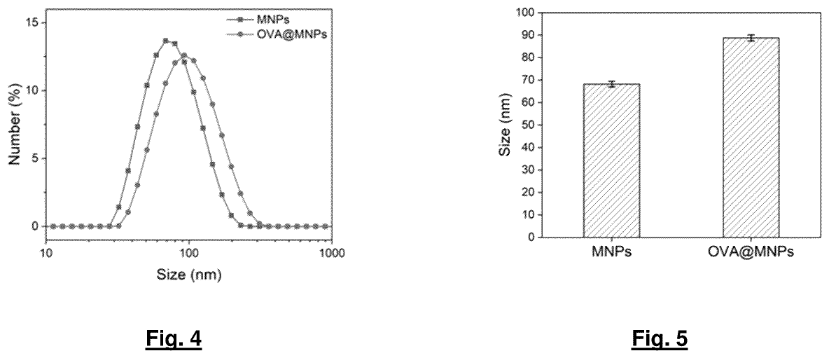

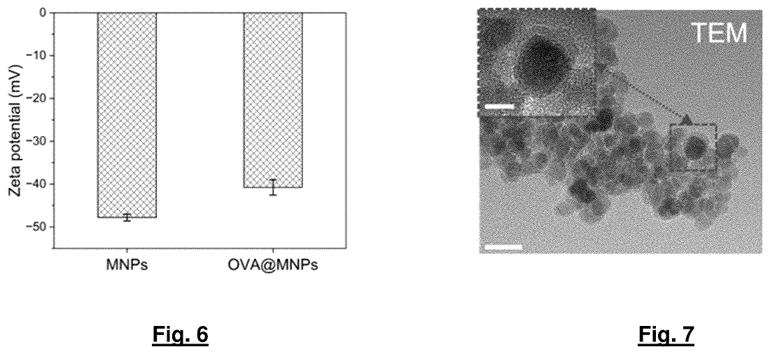

The synthesis processes and characterization results of the synthetic subunit vaccine, designated as OVA@MNPs, are shown in FIG. 3. Carboxyl-modified MNPs acted as carriers for the OVA antigen, undergoing loading through electrostatic adsorption in a MES buffer to form OVA@MNPs. Subsequently, a mixture solution of OVA@MNPs, GelMA, and LAP at a mass ratio of 20:100:25 was prepared, serving as the Inlet 2 material for subsequent microsphere fabrication, as depicted in FIG. 3. After the synthesis, dynamic light scattering (DLS) was employed to measure the nanoparticle size distribution of both MNPs and OVA@MNPs. The results revealed an increased hydrodynamic diameter from 68.23 nm to 88.74 nm following OVA loading (FIGS. 4 and 5). Additionally, the prepared OVA@MNPs were resuspended in deionized water, and the Zetasizer test results showed that antigen loading tuned the nanoparticle surface to less electronegative, shifting from −47.79 mV to −40.77 mV (FIG. 6). Table 1 illustrates the changes in the zeta potential of OVA under various pH buffer conditions, which informed the selection of an MES buffer for electrostatic adsorption when preparing OVA@MNPs.

TABLE 1
Zeta potential of OVA in different solvents

| | | | Zeta Potential |
|---|---|---|---|
| Solute | Solvent | pH of Solvent | [mV] |
| OVA | 0.1M MES | 4.3 | 12.28 |
| OVA | 1 × PBS | 7.4 | −12.85 |

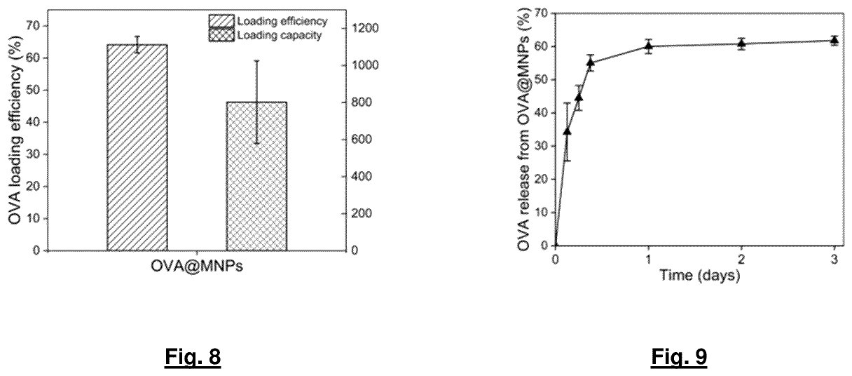

TEM image (FIG. 7) of the OVA@MNPs depicted a monodispersed, spherical formulation. OVA loading efficiency for OVA@MNPs reached 64.17% at an OVA/MNPs mass ratio of 1:4, with a maximum loading capacity of 802 mg per gram of MNPs, as shown in FIG. 8. Besides, the release of OVA from OVA@MNPs was investigated. OVA@MNPs underwent oscillation and ultrasonication in phosphate-buffered saline (PBS) for three days to facilitate OVA release. The release curve indicated that approximately 60% of OVA was released from the OVA@MNPs on the third day (FIG. 9).

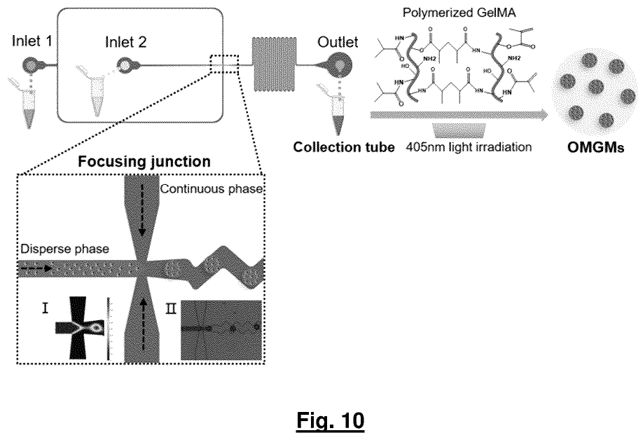

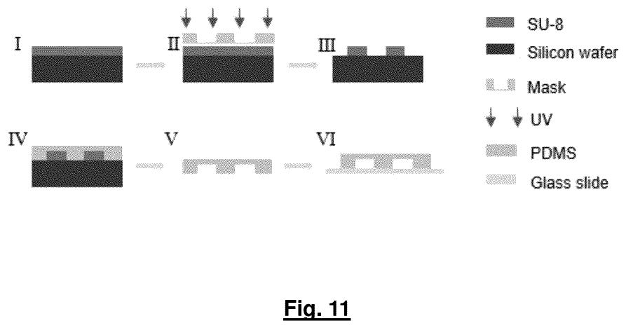

After the synthesis of OVA@MNPs, OMGMs were massively produced based on a microfluidic chip. FIGS. 10 to 18 outline the fabrication and characterization of OMGMs. A schematic drawing depicting the production of OMGMs using a microfluidic chip is provided in FIGS. 10 and 11. The process for fabricating the microfluidic chip includes:
Step I—spincoating SU-8 (a photoresist) on a silicon wafer with a height of 50 μm;
Step II—using a mask on the top of SU-8 to expose it under UV light after pre-baking;
Step III—developing the exposure SU-8 in developing reagent 1-methoxy-2-propanol acetate (PGMEA);
Step IV—solidifying/curing polydimethylsiloxane (PDMS) at 80° C.;
Step V—peeling off the cured PDMS; and
Step VI—bonding the cured PDMS to a glass slide.

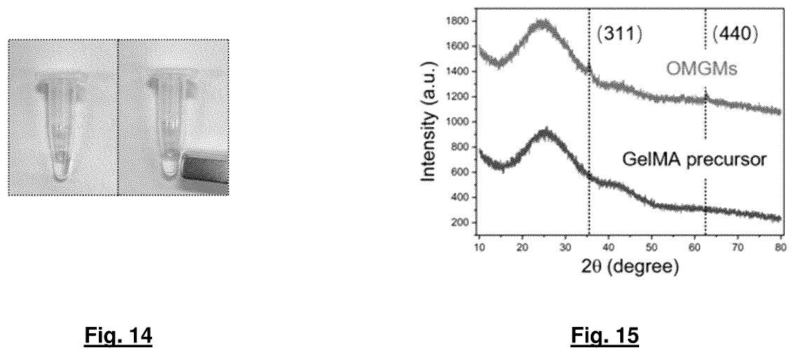

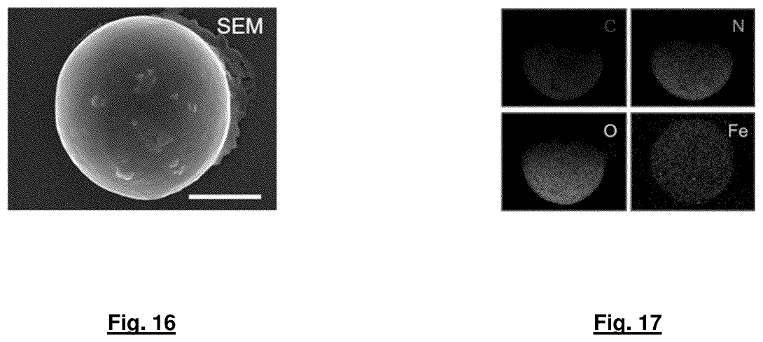

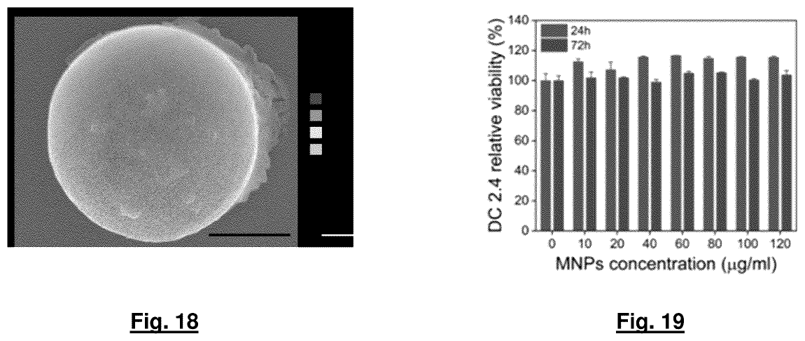

The photocurable disperse phase solution, to be introduced into the microfluidic chip through Inlet 2, consisted of GelMA, LAP, and OVA@MNPs, as illustrated in FIG. 3. The continuous phase solution, to be introduced into the microfluidic through Inlet 1, was prepared by blending mineral oil with surfactants (Span 80) to modulate the interfacial tension and viscosity of the solution. After injecting the solutions into the respective inlets, microspheres were generated by shearing the dispersed phase solution at the interface. The droplet size was readily adjustable by manipulating the flow rate, oil phase viscosity, and microfluidic channel dimensions. The rate of generating droplets using the microfluidic device was over 1,000 droplets per second. Afterward, the generated microspheres were transformed into OMGMs under the irradiation of a 405 nm light source for 2 min. After the microspheres were cured to obtain OMGMs, they were sonicated for 5 min to obtain uniformly dispersed OMGMs. Meanwhile, OMGMs expanded slightly owing to water absorption in hydrophilic conditions. Then, OMGMs were preserved in PBS and stored at 4° C. for various experiments, tests and applications. A partial view of the focusing junction area is shown in the enlarged view in FIG. 10. Through the simulation diagram (inset I of FIG. 10) and the actual microsphere generation diagram (inset II of FIG. 10), the correctness of the selected two-phase flow parameters was verified. The prepared OMGMs are depicted in FIG. 13, and the magnetic responsiveness of the OMGMs were confirmed by controlling multiple OMGMs by placing a square magnet on the outside of a tube (FIG. 14). Furthermore, XRD analysis was performed on the GelMA precursor and OMGMs (see results shown in FIG. 15). The peak positions at (311) and (440) representing typical crystalline planes of Fe₃O₄ were found, further indicating the success of encapsulating OVA@MNPs in OMGMs. FIG. 16 shows SEM images portraying the overall structure and surface of OMGMs. EDS results are shown in FIG. 17. FIG. 18 reveals a uniform distribution of OVA@MNPs (depicted in light green) within OMGMs.

### In Vitro and In Vivo Biodegradation and Toxicity

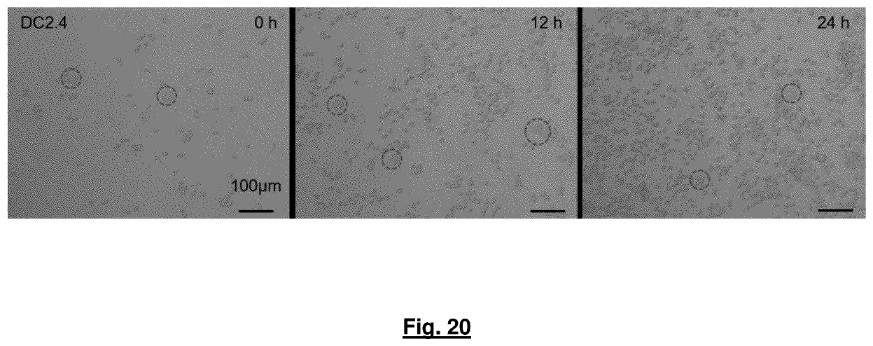

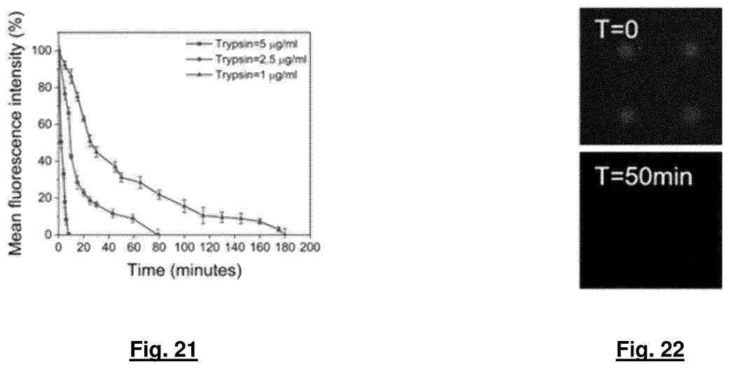

After OVA@MNPs synthesis and OMGMs fabrication, in vitro and in vivo biodegradation and toxicity were further investigated. The in vitro and in vivo biodegradation of OMGMs relies on the enzymatic degradability of GelMA. DC2.4 Mouse Dendritic cells were utilized for the in vitro assays. It was confirmed that the carboxyl-modified MNPs selected as preparation materials were not cytotoxic. As shown in FIG. 19, DC2.4 cell viability remained above 85% after one day (24 h) and three days (72 h) of coculturing with varying concentrations of MNPs. In comparison, the viability of the treatment by the highest dose (120 μg mL⁻¹ MNPs) remained above the 80% viability level. Furthermore, the culture status of DC2.4 cells remained stable after coculturing with OMGMs for 12 and 24 h, as shown in FIG. 20. In particular, FIG. 20 shows the stable status of DC2.4 cells after 12 h of coculturing with the OMGMs, and no suspended apoptotic cells were found. Subsequently, the biodegradation of OMGMs was assessed under different concentrations of trypsin-EDTA (TE), namely, 1, 2.5, and 5 μg mL⁻¹ (FIG. 21). Degradation lasted for approximately 3 h with a 1 μg mL⁻¹ TE solution, while such occurred within about 10 min with a 5 μg mL⁻¹ TE solution. The fluorescence comparison chart after degradation for 50 min under 2.5 μg mL⁻¹ TE is shown in FIG. 22, in which the green fluorescence represents the microspheres. After the degradation of OMGMs, degradation products equivalent to 0, 1×10⁴, 2×10⁴, and 3×10⁴ OMGMs were cultured with DC2.4 cells. After culturing for 24 h and 72 h, the relative viability of the cells was maintained above 80%, as shown in FIG. 23, further confirming the safety of OMGMs. Overall, in vitro experimental outcomes indicated that the biodegradation and toxicity of the OVA@MNPs and OMGMs did not affect cell culturing.

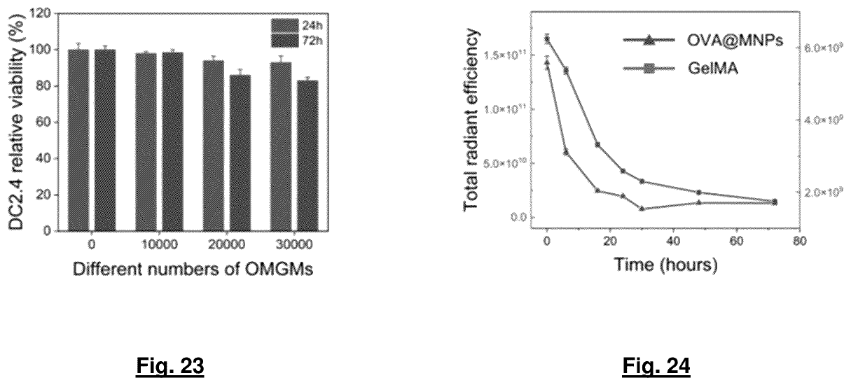

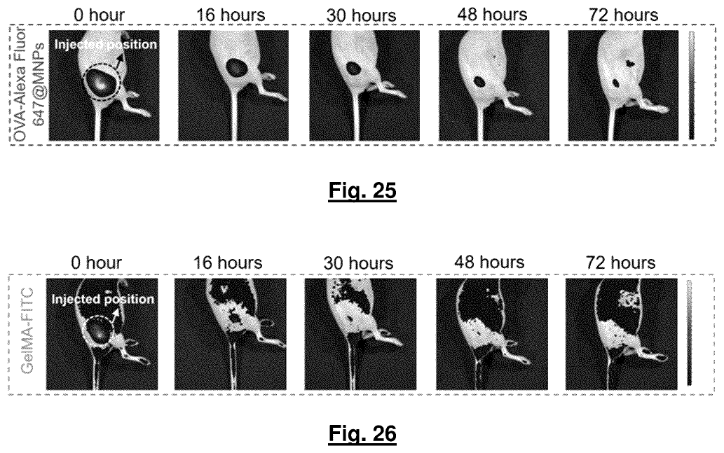

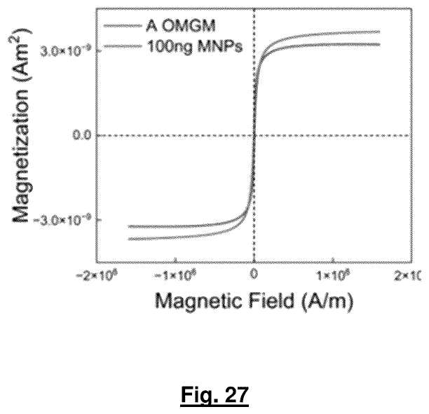

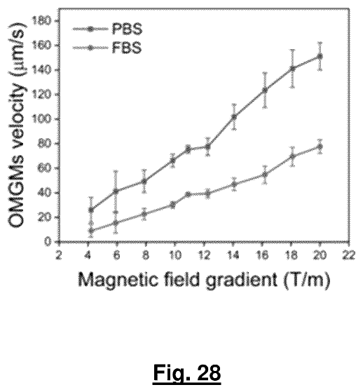

Afterward, nude mice were used as animal models for subsequent in vivo experiments. Red fluorescent OVA@MNPs were produced by conjugating OVA with Alexa Fluor 647, while Gelatin Methacryloyl-Fluorescein isothiocyanate (GelMA-FITC) was utilized to monitor hydrogel biodegradation. Alexa Fluor 647 and FITC were chosen as fluorescence choices for observing OVA@MNPs and the GelMA hydrogel of OMGMs structures, respectively. OMGMs were injected subcutaneously into the right dorsal area, and the mice were monitored at various intervals using an IVIS instrument. With the observation for three days, changes in the total radiant efficiency of the injected area were monitored to draw the curves in FIG. 24. Besides, the biodegradation process was monitored by observing the intensity of Alexa Fluor 647, as shown in FIG. 25, wherein the fluorescence signal of Alexa Fluor 647-OVA gradually weakened after 72 h. Similarly, FIG. 26 illustrates the biodegradation process by observing the intensity of GelMA-FITC, which also weakened after 72 h. When it was observed through the IVIS system that the signals were all caused by skin, the OMGMs were deemed entirely degraded. Then, the mice were monitored for a month, and no abnormal death was found. In vitro and in vivo experiments confirmed the biodegradation and nontoxicity of OMGMs.

### Characterization and Magnetic Actuation of OMGMs

After verifying the fundamental characterization of the synthesized OVA@MNPs and fabricated OMGMs, the magnetic driving property of OMGMs was further verified. Using MNPs to prepare the subunit vaccine OVA@MNPs allows OMGMs to exhibit motility under a magnetic field. The soft magnetic behavior of the OMGMs is evident from the hysteresis loop depicted in FIG. 27. Despite the superparamagnetic nature of OVA@MNPs, OMGMs exhibited low coercivity (~3.8 kA m⁻¹) due to MNPs agglomeration within GelMA. Subsequently, an electromagnetic coil system was employed to regulate the direction and gradient of the external magnetic field, facilitating the drag propulsion of OMGMs. FIG. 28 illustrates the velocities of OMGMs in PBS and fetal bovine serum (FBS) under a magnetic field with a gradient ranging from 0 to 20 T m⁻¹. The electromagnetic coil system for driving the OMGMs was designed with a magnetic field strength greater than 150 mT and a working space larger than 160 mm.

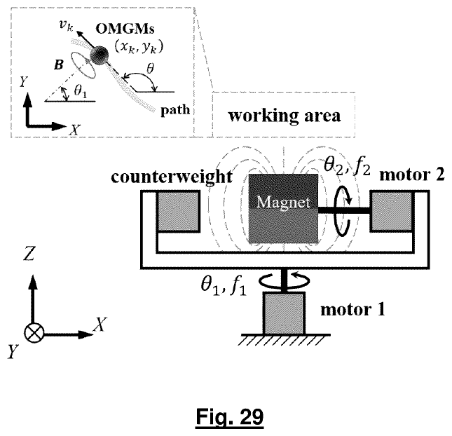

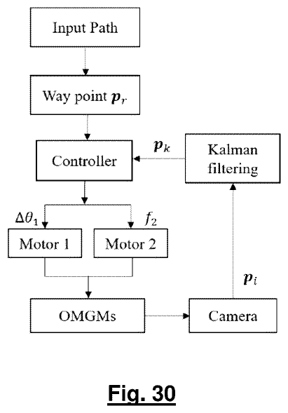

Additionally, a rotating permanent magnet system (such as one shown in FIG. 29) was used to generate a controllable rotating magnetic field, enabling the rolling propulsion of the OMGMs. The magnetic field originates from a cylindrical NdFeB magnet (N52 Grade) driven by two orthogonally arranged stepper motors. The motor 2 rotates to generate a rotating magnetic field with frequency f₂. The rotation axis of the magnetic field is in the plane XY and can be adjusted by motor 1 rotating around the Z axis. The magnetic moment of OMGMs aligns to the magnetic field direction B during rolling until the rotation frequency goes beyond the step-out frequency. Thus, the motion of the rolling OMGMs can be simplified as a unicycle model. Considering the low-Reynolds number liquid environment, kinematic model of the magnetic OMGMs expresses

$$\{ \begin{matrix}{\dot{x}}_{k} ( t ) = {v}_{k} ( {f}_{2} ( t ) ) cos ( θ ( t ) ) \\ {\dot{y}}_{k} ( t ) = {v}_{k} ( {f}_{2} ( t ) ) sin ( θ ( t ) ) \\ \dot{θ} ( t ) = ω ( t )\end{matrix} ,$$

where νₖ and ω are the forward and steering velocity, and xₖ(t) and yₖ(t) are orthogonal components of the OMGMs position pₖ. νₖ is proportional to the rotation frequency f₂ (t) of the magnetic field when this frequency is below step-out frequency and θ(t) denotes the forward direction of the OMGMs.

A closed-loop motion control strategy is developed to ensure the OMGMs can be actuated along the preset path, as FIG. 30 depicts. To enable the magnetic microsphere to roll along a desired path, a motion control system has been developed. The desired path is discretized into N way points pr={pr, i|i=1, . . . , N}. The current position pₖ of microsphere is obtained by using a template-matching-method-based algorithm, and Kalman filter is used to reduce the impact of disturbance. The rotation frequency f₂ of motor 2 and angle change Δθ1 of motor 1 determines the motion status of the magnetic microsphere. Thus, a p-type close-loop controller is taken as follow:

$$\{ \begin{matrix}{Δθ}_{1} = \{ \begin{matrix}min ( {k}_{1} ( {θ}_{r} - {θ}_{k} ) , {θ}_{s} ) & {θ}_{r} - {θ}_{k} > 0 \\ max ( {k}_{1} ( {θ}_{r} - {θ}_{k} ) , - {θ}_{s} ) & {θ}_{r} - {θ}_{k} \leq 0\end{matrix} \\ {f}_{2} = min ( {k}_{2} ‖ {p}_{r} - {p}_{k} ‖ , {f}_{s} )\end{matrix}$$

where θᵣ and θₖ are the reference forward direction and current forward direction, and k₁ and k₂ are the proportional parameters of the controller. θₛ is the upper limit of angle change of motor 1 and fₛ is the upper limit of the frequency of motor 2, which depends on the sampling time Tₛ and mechanical properties of motors. At time tⱼ, the magnetic microsphere moves to p_{r,i}=(x_{r,i}, y_{r,i}), θᵣ and θₖ express:

$$\{ \begin{matrix}{θ}_{r} = {tan}^{- 1} \frac{{y}_{\text{?}} - y ( {t}_{j} )}{{x}_{\text{?}} - x ( {t}_{j} )} \\ {θ}_{k} = {tan}^{- 1} \frac{y ( {t}_{j} ) - y ( {t}_{j - 1} )}{x ( {t}_{j} ) - x ( {t}_{j - 1} )}\end{matrix} ,$$

$$\text{?} \text{indicates text missing or illegible when filed}$$

where x(tⱼ), y(tⱼ) and x(tⱼ₋₁), y(tⱼ₋₁) are the microsphere position pₖ at time tⱼ and tⱼ₋₁ respectively. When position error eₚ=∥p_{r,i}-p_{k,i}∥ drops below εₚ, the magnetic microsphere rolls to next target point p_{r+1,i} until i reaches N.

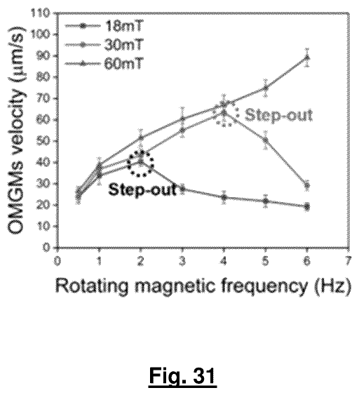

The rotating field provides a magnetic torque to OMGMs, allowing them to rotate with the magnetic field and roll forward. The frequency and intensity of the rotating magnetic field determine the rolling velocity of the OMGMs. Subsequently, rotating magnetic fields with 18, 30, and 60 mT intensity were applied at various rotational frequencies from 0.5 Hz to 6 Hz to actuate the OMGMs (see FIG. 31). As the external magnetic field strength increased, the speed of the OMGMs increased until it reached the maximum speeds of 38 and 57 μm s⁻¹ at (2 Hz, 18 mT) and (4 Hz, 30 mT), respectively. The frequency at which the maximum speed is induced is also called the step-out frequency and depends on the intensity of the magnetic field. When the frequency exceeded the step-out frequency, the speed gradually decreased.

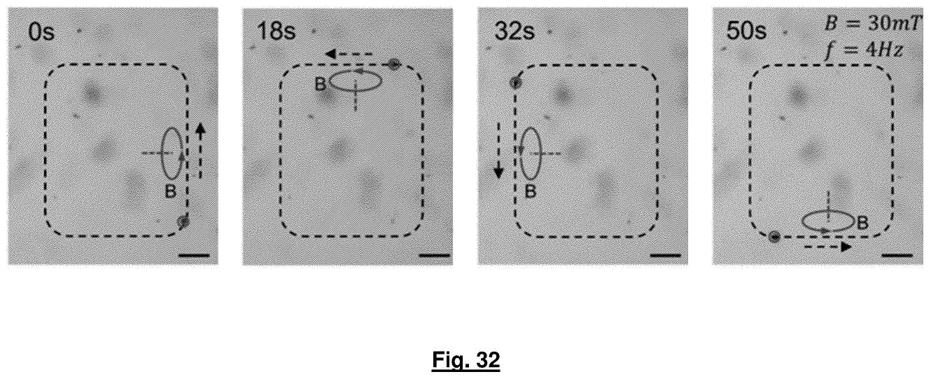

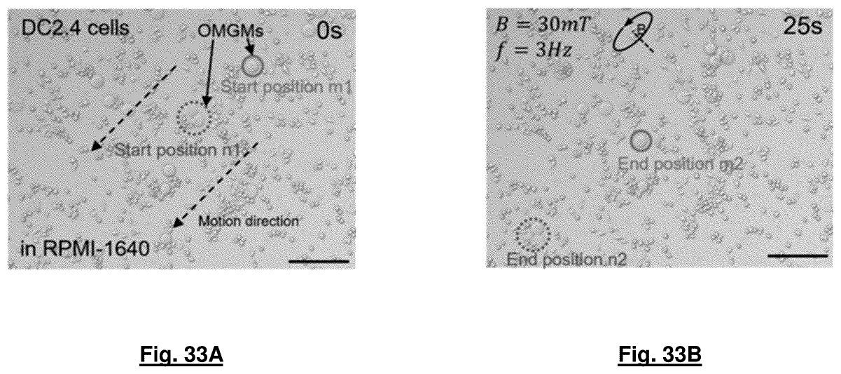

Furthermore, FIG. 32 shows the process of OMGMs tracking a rectangular path under a rotating magnetic field (4 Hz, 30 mT). Afterward, the OMGMs were further subjected to movement experiments in the DC2.4 cell culture environment with Roswell Park Memorial Institute (RPMI) 1640 medium, as shown in FIGS. 33A and 33B. Under a rotating magnetic field of (3 Hz, 30 mT), the OMGMs moved along the direction indicated by the dashed arrows in FIG. 33A. The movement starts and end positions of the two groups of OMGMs are marked as (m1, m2) and (n1, n2), respectively. The solid circle denotes the first group of OMGMs, and the dashed circle represents the second group of OMGMs. The experimental results demonstrate that OMGMs exhibit excellent motion performance under two magnetic fields, providing different driving solutions for different environments.

### In Vitro Cellular Uptake, Cross-Presentation, and Immune Response

In vitro cellular uptake, cross-presentation, and induced immune response performances of OVA@MNPs loaded with OMGMs were examined, for the purpose of assessing potential use of OMGMs of the present invention for immune therapy.

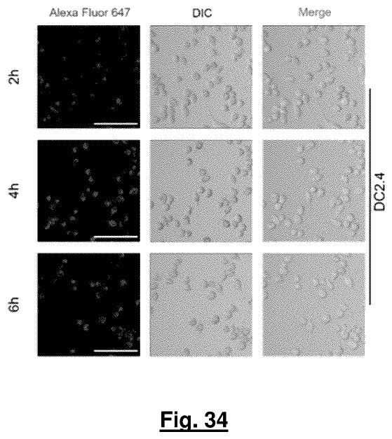

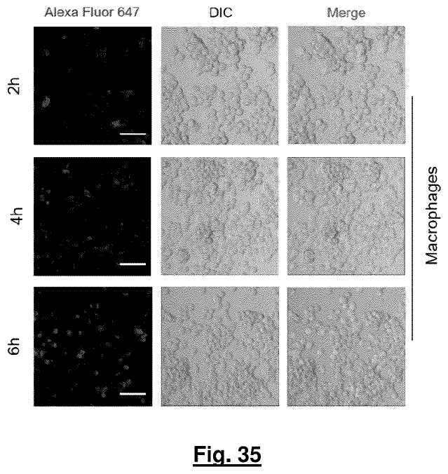

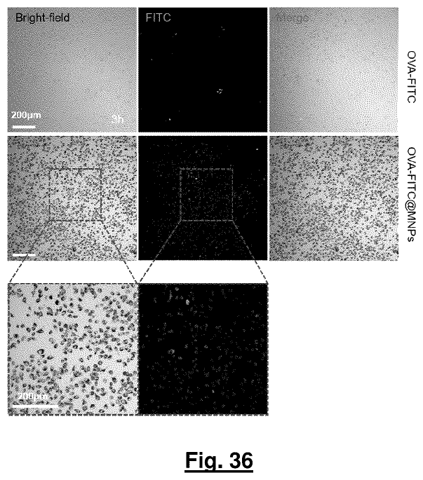

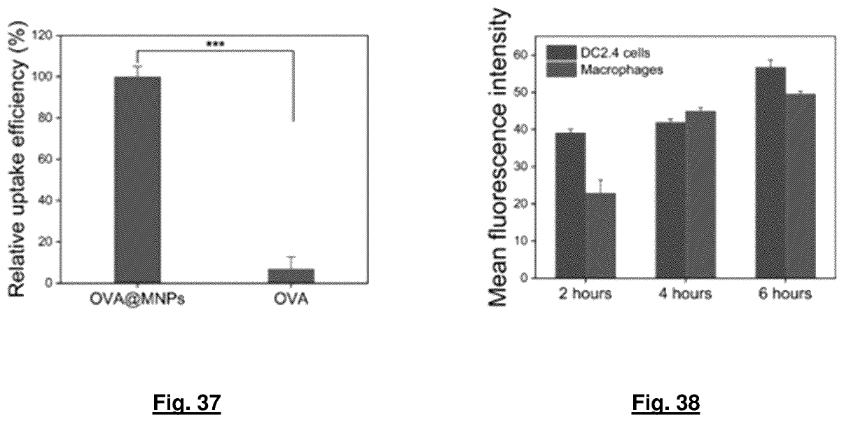

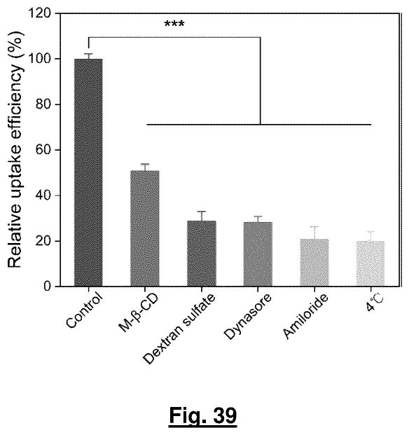

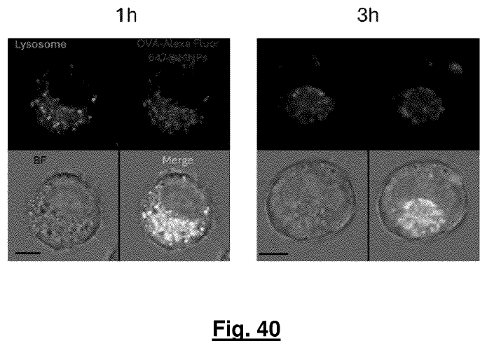

Alexa Fluor 647 and FITC fluorescence were used to label OVA and assess the internalization performance of OVA@MNPs in DC2.4 cells and RAW264.7 macrophages. Endocytosis helps APCs internalize OVA@MNPs, thereby improving the uptake efficiency of OVA. The uptake images of DC2.4 cells (FIG. 34) and RAW264.7 macrophages (FIG. 35) illustrate the excellent cellular uptake of OVA@MNPs by APCs. Afterward, OVA-FITC and OVA-FITC@MNPs loaded with an equivalent amount of OVA were added to the DC2.4 culture medium for 3 h. The uptake image (FIG. 36) shows the excellent uptake of OVA@MNPs, as evidenced by the increase in the black area representing the MNPs and the green fluorescence representing OVA. Flow cytometry analysis revealed a 20-fold increase in the relative uptake efficiency of DC2.4 cells, as shown in FIG. 37. Further confirmation of the exceptional uptake efficiency of OVA@MNPs was provided by the quantitative mean fluorescence intensity of DC2.4 cells and RAW264.7 macrophages after 2-6 h of culturing, as illustrated in FIG. 38. To investigate the internalization mechanism of OVA@MNPs, DC2.4 cells underwent pretreatment with various inhibitors to impede specific endocytic pathways. As depicted in FIG. 39, the uptake efficiency decreased by 80% when exposed to a temperature of 4° C., indicating the energy-dependent nature of OVA@MNPs internalization. Besides, the presence of dextran sulfate led to a 70% reduction in uptake, suggesting that OVA@MNPs primarily entered cells through scavenger receptor-mediated endocytosis. Additionally, inhibition by dynasore implied the involvement of dynamin-dependent endocytic pathways in the internalization of OVA@MNPs. Furthermore, the uptake of OVA@MNPs was significantly impacted by methyl-β-cyclodextrin and amiloride, affirming the participation of caveolae/lipid raft-mediated endocytosis and micropinocytosis, respectively. Furthermore, the separation of red fluorescent signal of OVA-Alexa Fluor 647@MNPs and green fluorescence from lysosomes indicated the escape of OVA@MNPs from the endolysosomes (FIG. 40).

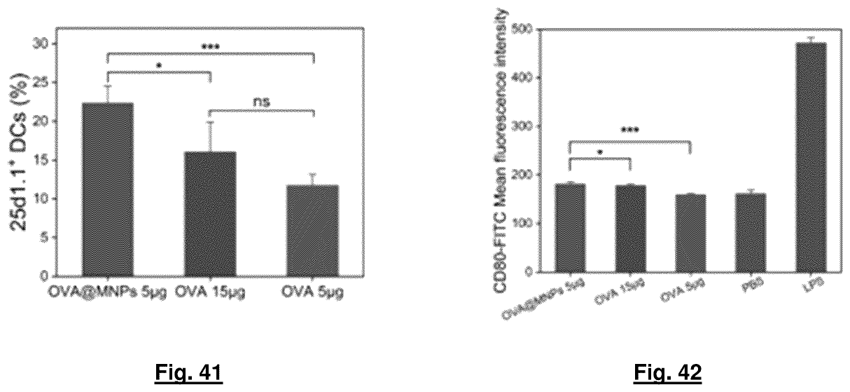

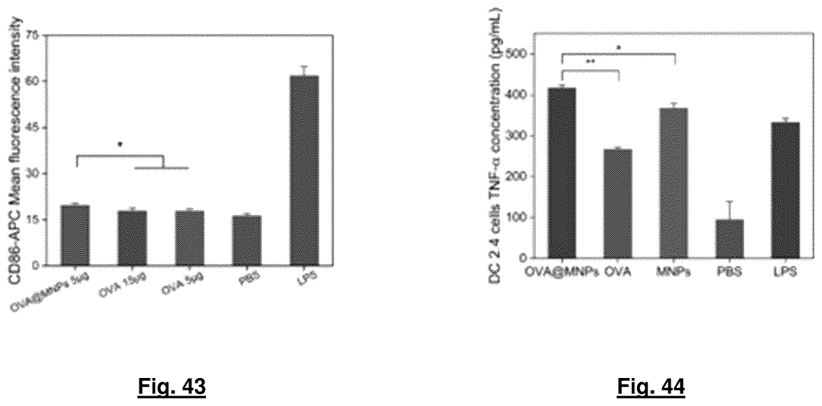

Next, antigen cross-presentation was assessed by analyzing the H-2Kb-SIINFEKL complex on DC2.4 cell surfaces via flow cytometry labeled with the monoclonal antibody 25d1.16. Compared with the free OVA group, the proportion of 25d1.16 positive cells in OVA@MNPs loading with 5 μg OVA doubled (FIG. 41), and the results also significantly exceeded those of the 15 μg free OVA group. Moreover, to explore the potential of OVA@MNPs in stimulating the maturation of primary cells, the costimulatory molecules CD80 and CD86 levels were quantified in BMDCs. OVA@MNPs exhibited a significant upregulation in the expression of CD80 and CD86 factors compared to PBS and reached a level higher than that of the 15 μg free OVA, indicating their potential adjuvant advantage (FIGS. 42 and 43). The in vitro experiment demonstrated that OVA@MNPs enhanced cellular uptake and cross-presentation efficiency.

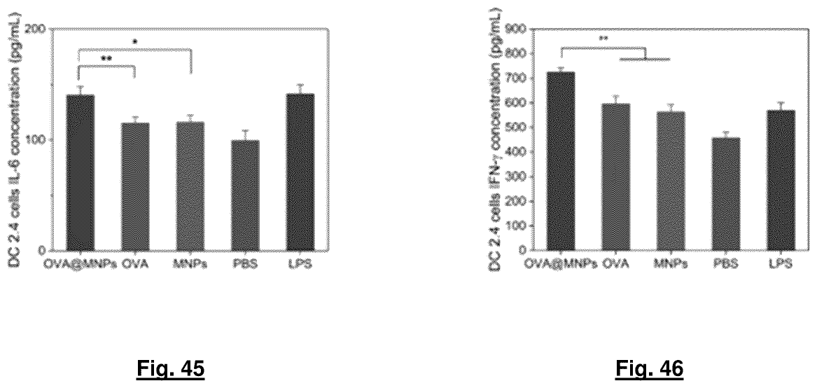

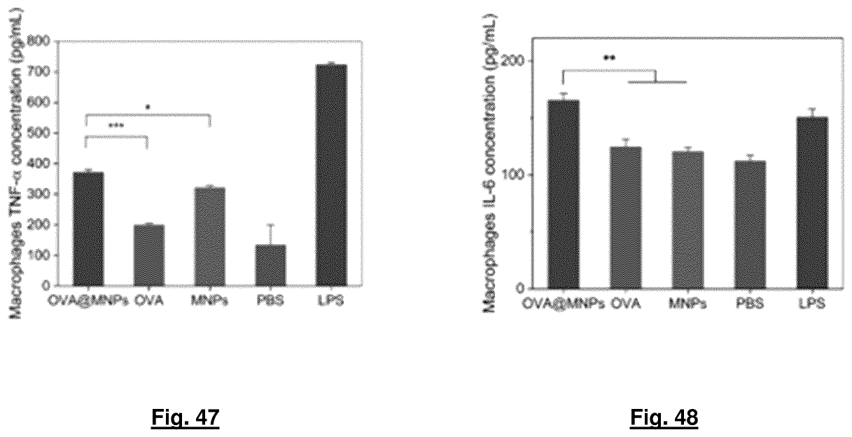

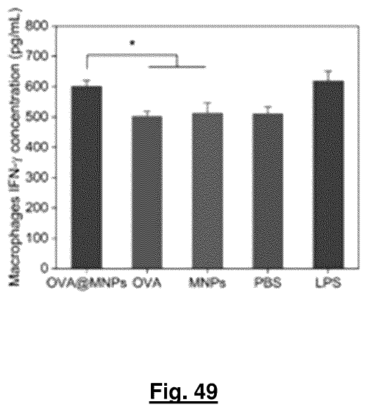

Furthermore, DC2.4 cells and RAW264.7 macrophages were cultured with OVA@MNPs to verify their immunostimulatory property. After 24 h of incubation, cell culture supernatants were collected for enzyme-linked immunosorbent assays (ELISA) to measure pro-inflammatory cytokine expression levels, including interferon-gamma (IFN-γ), interleukin 6 (IL-6), and tumor necrosis factor-alpha (TNF-α). These molecules are essential to the immune system. As shown in FIGS. 44 to 46 and FIGS. 47 to 49, OVA@MNPs considerably improved the concentrations of secreted IFN-γ, IL-6, and TNF-α in the cell supernatants compared to the four control treatments. Compared with OVA-treated DC2.4 cells, the secretion levels of immune response factors such as TNF-α, IL-6, and IFN-γ with DC2.4 cells treated by OVA@MNPs were increased by approximately 1.7, 1.2, and 1.3 times, respectively. Besides, compared with OVA-treated RAW264.7 macrophages, the secretion levels of immune response factors such as TNF-α, IL-6, and IFN-γ with RAW264.7 macrophages treated by OVA@MNPs were increased by approximately 2, 1.4, and 1.3 times, respectively. OVA@MNPs loaded with OMGMs effectively stimulated better cellular uptake, cross-presentation, and immune response.

### In Vivo Draining and Targeting of the Lymph Nodes

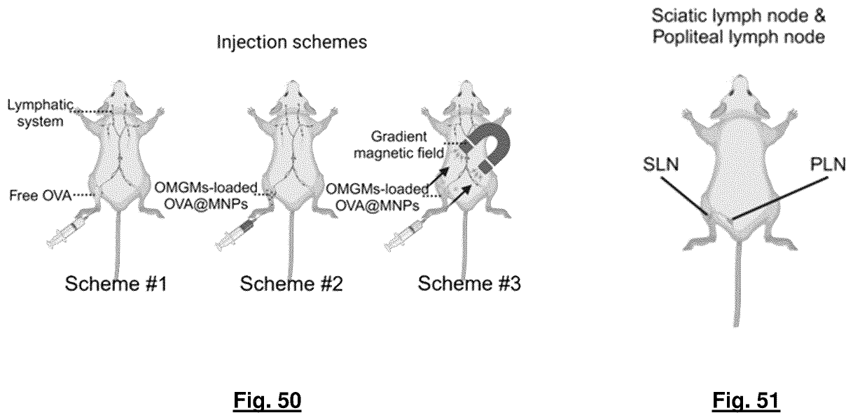

As lymph nodes serve as the site of origin for adaptive immune responses, the lymphatic drainage and targeting capabilities of OMGMs-loaded OVA@MNPs were evaluated. Three injection schemes were designed to verify that the proposed OMGMs-loaded OVA@MNPs have targeted drainage effects with the external gradient magnetic field (FIG. 50). Scheme #1 and #2 served as control groups compared to Scheme #3 to observe the lymphatic drainage advantages of the prepared OMGMs-loaded OVA@MNPs and the enhanced effect of the added magnetic field. Free Alexa Fluor 647-OVA and Alexa Fluor 647-OVA@MNPs were prepared and administered via subcutaneous injection into the hind foot pads of mice. In addition, external magnetic field application method of OMGMs-loaded OVA@MNPs was described with reference to the schematic diagram in FIG. 50 and the experimental section. In order to facilitate the subsequent observation of the signal intensity of the main lymph node locations in mice, the approximate locations of the sciatic lymph node (SLN) and popliteal lymph node (PLN) were illustrated by drawing the lymph node distribution map of the mouse lymphatic system in FIG. 51.

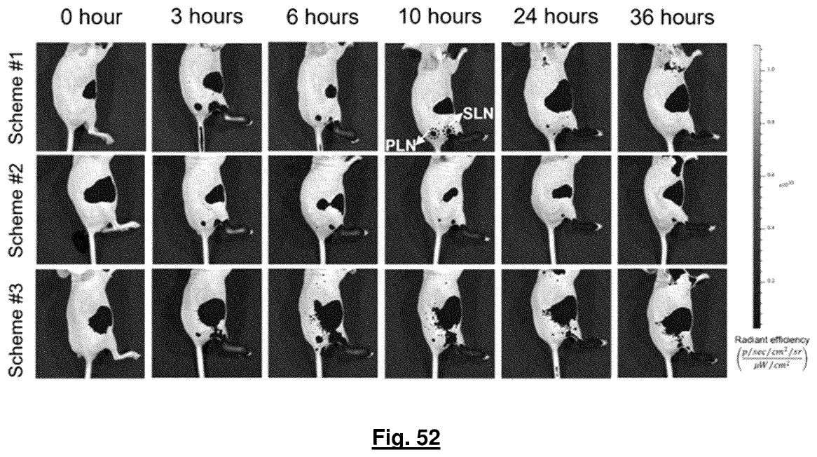

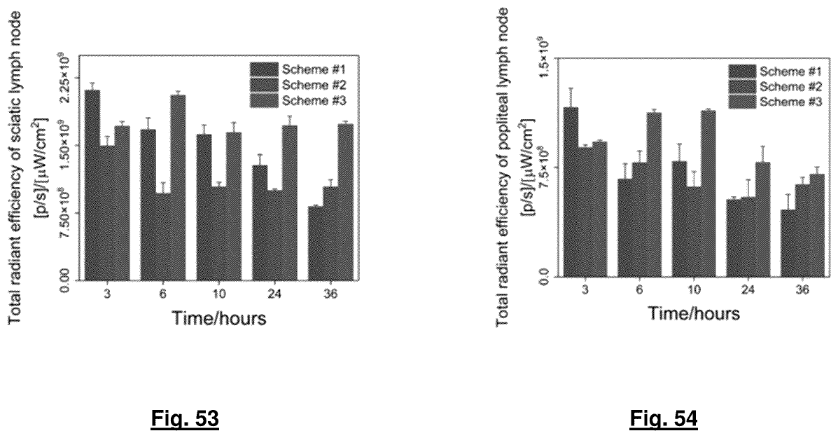

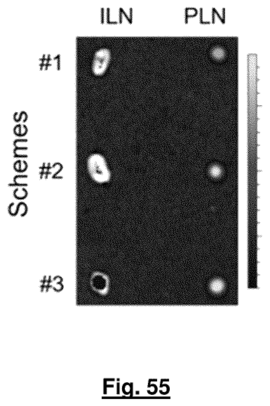

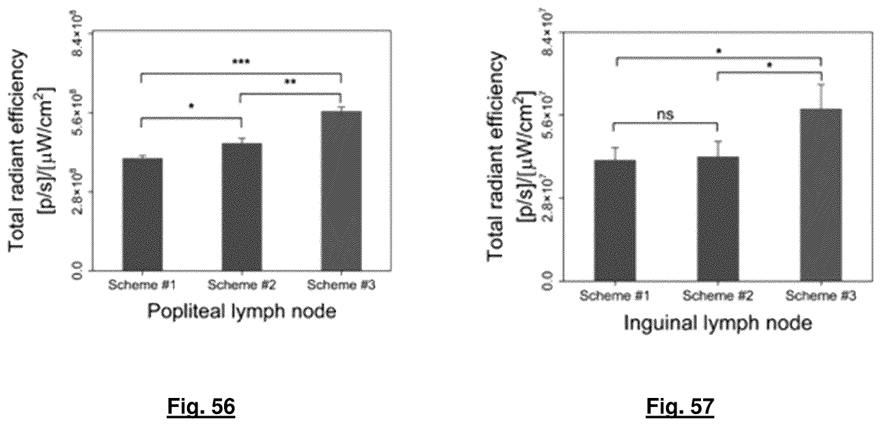

Then, the IVIS instrument was used to visually photograph the mice at different time points over the next 36 h to record the signal intensity changes of lymph nodes at different locations (FIG. 52). In the free OVA group, a fluorescence signal was initially detected in the PLN and SLN, but it rapidly weakened post-injection. After 36 h, the signal intensity at the SLN and PLN in the free OVA group was weaker than the signal intensity at the exact location in the OVA@MNPs and OMGMs groups, indicating that OMGMs and OVA@MNPs helped more antigens reside in lymph node locations rich in APCs. The quantitative changes in the total radiation efficiency of SLN and PLN are shown in FIGS. 53 and 54. After 36 h, the efficiency at the SLN and PLN locations in the OMGMs group was 2.3 times and 1.5 times higher than that in the OVA group, respectively. Besides, the signal intensity notably increased in the sciatic and popliteal lymph nodes, peaking 6 h post-injection. After 36 h of visualization, the mice were sacrificed, and the PLN and inguinal lymph node (ILN) were collected to obtain ex vivo images. Results showed a significantly higher accumulation of OMGMs and OVA@MNPs in these lymph nodes than free OVA (FIG. 55). Total radiant efficiency was analyzed based on the images of the collected lymph nodes, as shown in FIGS. 56 and 57. The quantitative results revealed that magnetically driven OMGM enhanced the draining and targeting efficacy of the lymph nodes.

### Immune Response and Ability to Inhibit Tumor Growth

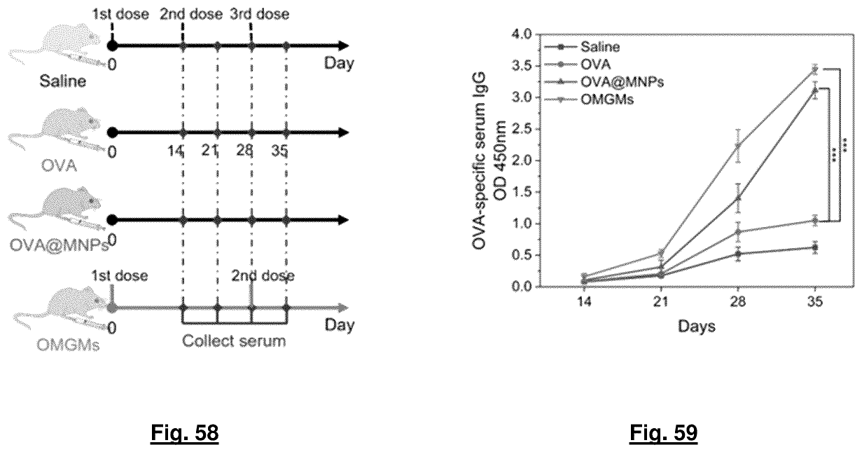

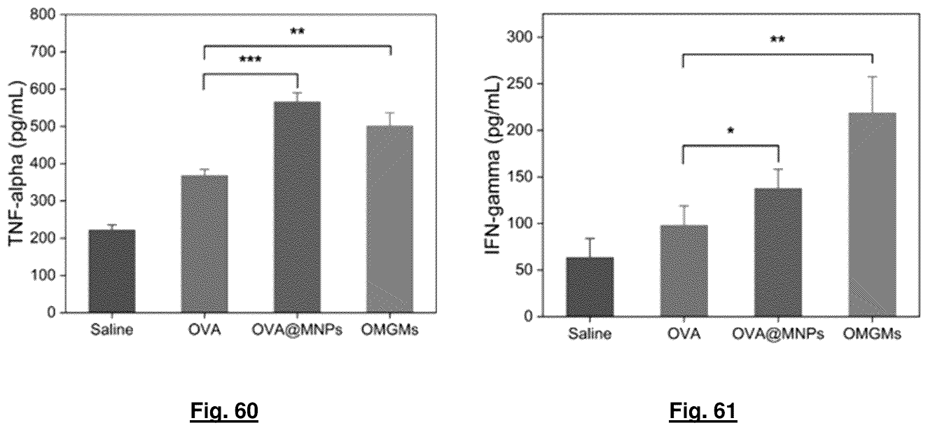

Mouse vaccination and blood collection detection experiments have been designed (see FIG. 58). To prove the immune advantage of the sustainable-release degradation of OMGMs, a single booster immunization regimen (i.e., a total of two vaccine injections) was used for the OMGMs group in the vaccination experiment. In contrast, two booster immunization regimens were used for the OVA and OVA@MNPs groups (i.e., a total of three vaccine injections). Specifically, mice received subcutaneous injections of 10 μg OVA and OVA@MNPs loaded with 10 μg OVA on days 0, 14, and 28, constituting a regimen with two immune booster injections. Alternatively, mice were administered OMGMs containing 20 μg OVA and 10 μg OVA on days 0 and 28, representing a regimen with only one immune booster injection. Subsequently, mandibular blood sampling collected mouse sera at different time points to detect immunoglobulin G (IgG) antibody responses on days 14, 21, 28, and 35 to assess the level of humoral immune response elicited. IgG antibodies in the saline and OVA groups peaked on day 28. In contrast, antibody levels in the OMGMs groups continued to rise until day 35 (FIG. 59). Compared to the free OVA, OMGMs induced considerably higher antibody responses. OMGMs exhibited ideal antibody performance, followed by the triple-immunized OVA@MNPs. The results indicated that the OVA@MNPs and biodegradable OMGMs remarkably enhanced the immunogenicity of OVA, and the results of a single booster injection were not worse than those of two booster injections. Otherwise, a panel of pro-inflammatory cytokines was determined by serum ELISA assay to explore further the potential immune-activating properties of the OMGMs. FIGS. 60 and 61 revealed secretion levels of TNF-α and IFN-γ, pivotal cytokines triggering early immune responses, in the serum of mice treated with OVA@MNPs and OMGMs. The IFN-γ and TNF-α secretion levels in the OMGMs group were increased by about 2.8 times and 1.4 times, respectively, compared with the OVA group. The results further demonstrated that a sustained-release subunit vaccine of biodegradable OMGMs induced enhanced immune responses.

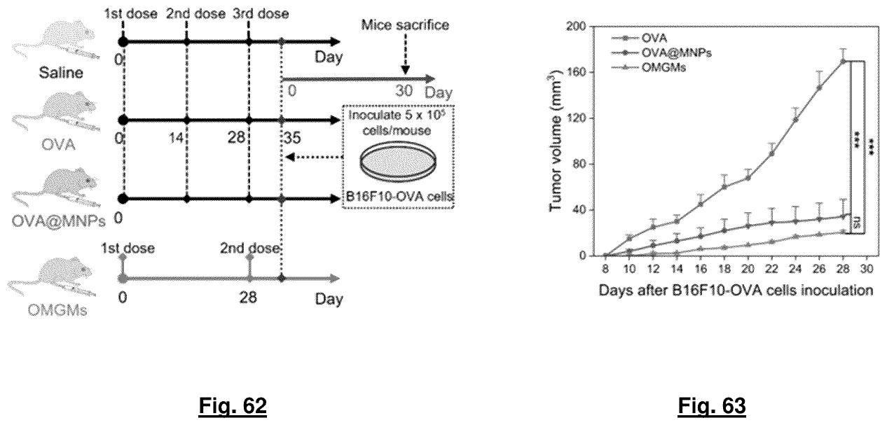

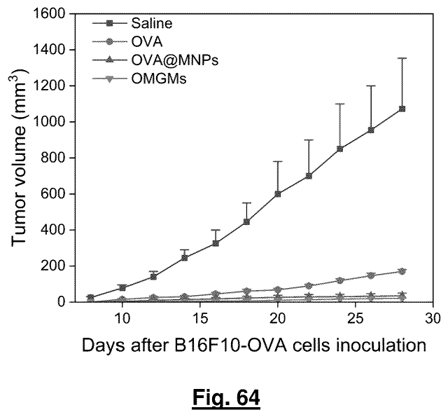

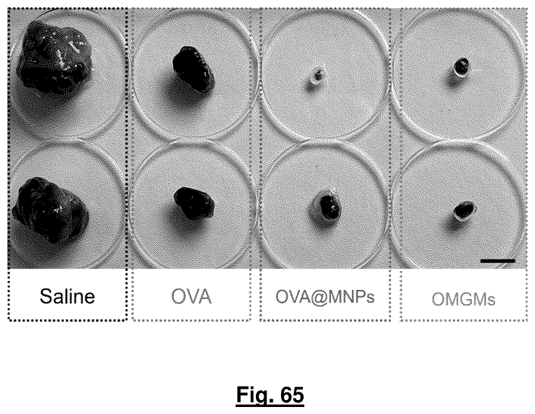

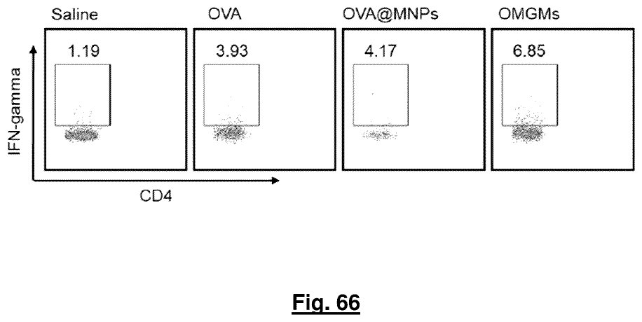

A tumor challenge experiment after mouse vaccination (FIG. 62) was designed. The antitumor vaccine potential of OMGMs was investigated to assess the ability to inhibit tumor growth. In this part of the in vivo experiment, the same immunization design as the vaccination experiment described above was used. Mice received subcutaneous injections of 10 μg OVA and OVA@MNPs loaded with 10 μg OVA on days 0, 14, and 28, constituting a regimen with two immune booster injections. Alternatively, mice were administered OMGMs containing 20 μg OVA and 10 μg OVA on days 0 and 28, representing a regimen with only one immune booster injection. On day 35, mice were subcutaneously inoculated with 5×10⁵ B16F10-OVA cells on the right posterior side. Twenty-eight days after melanoma cells were inoculated into mice, FIG. 65 shows tumor images of mice in the OVA, OVA@MNPs, and OMGMs groups after tumor peeling, and prove that the tumor volume of the OMGMs group was the smallest. One month after B16F10-OVA cell inoculation, the tumor growth of all mice was recorded in detail using vernier calipers and the tumor volume was calculated. Detailed tumor size growth curves are shown in FIG. 63. The OMGMs formulation showed a much stronger inhibition potency to tumor growth compared with the free OVA and OVA@MNPs formulations. Additionally, FIG. 64 provides the complete tumor growth curve after cell inoculation.

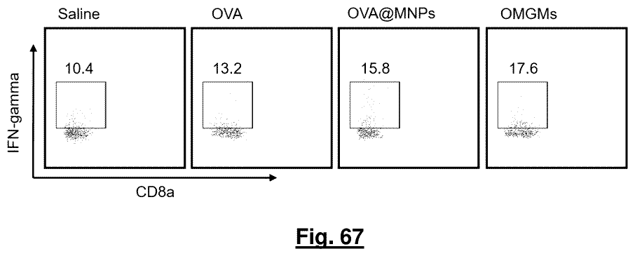

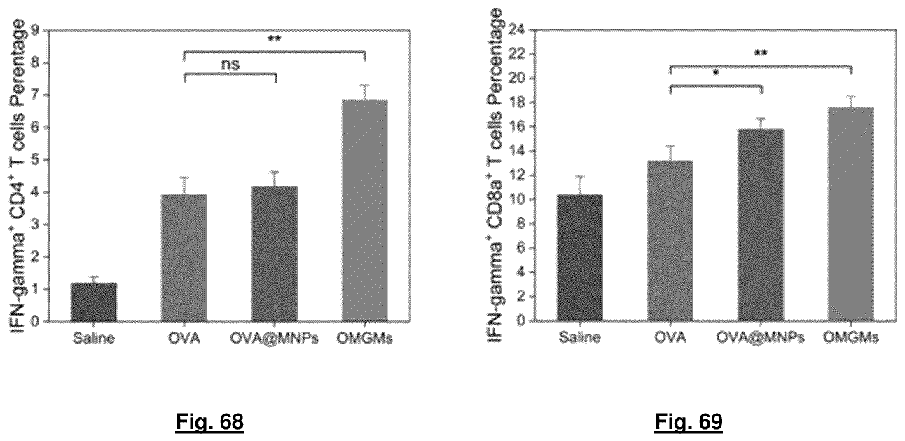

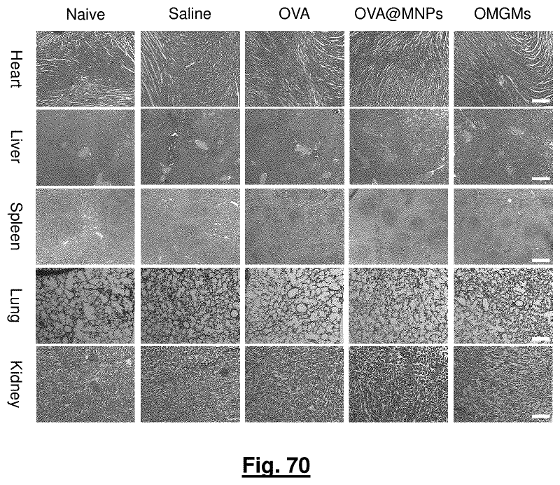

Subsequently, the CD4⁺ and CD8a⁺ T cell responses induced by OMGMs was evaluated by collecting splenocytes from vaccinated mice on day 30 and subjecting them to ex vivo culture and restimulation. Further immune responses elicited by the treatments were assessed by analyzing CD8a⁺ IFN-γ⁺ T and CD4+ IFN-γ⁺ T cells in the spleens of the mice. The amounts of CD4⁺ IFN-γ⁺ T and CD8a+ IFN-γ⁺ T cells in the spleens of the mice in the OMGMs group were higher than those in the spleens of the mice in the other groups (see FIGS. 68 and 69), which is critical for tumor inhibition, supporting the potential of OMGMs as a promising antitumor subunit vaccine platform. Furthermore, flow cytometry data of the percentages of CD4⁺ IFN-γ⁺ T and CD8a⁺ IFN-γ⁺ T cells in the OMGMs group is shown in FIGS. 66 and 67.

Finally, the systemic and local toxicities of OMGMs were assessed to confirm biosafety as a subunit vaccine delivery strategy. Seven days after immunization completion, hematoxylin and eosin (H&E) staining was performed on the hearts, livers, spleens, lungs, and kidneys of immunized mice (FIG. 70). Notably, no apparent abnormalities were found in the mice treated with OVA, OVA@MNPs, or OMGMs, indicating the biosafety of the subunit vaccine delivery strategy and the administered doses.

This present invention provides a novel method of utilizing biodegradable magnetically driven microspheres (OMGMs) to deliver subunit vaccines (antigens) and enhanced immunotherapy efficiently. OMGMs demonstrated the potential for mass production and served as a platform for efficiently delivering subunit antigens.

OVA@MNPs loaded with the model protein OVA were successfully formulated, exhibiting a diameter below 100 nm. Focus-flowing microfluidic droplet generation technology produced large numbers of OMGMs at a production rate reaching over 1,000 per second. This capability is critical for practical use and commercial advancement of microstructures in biomedical applications. Subsequently, comprehensive characterization and verification of the prepared subunit vaccines and OMGMs were conducted utilizing SEM, TEM, XRD, and DLS techniques. Furthermore, to assess the biocompatibility and biodegradability of the synthesized OVA@MNPs and fabricated OMGMs, a series of in vitro and in vivo experiments were performed, employing cells and mice, respectively. The results from these experiments demonstrated the favorable biocompatibility and biodegradability of both OVA@MNPs and OMGMs, establishing their suitability for biomedical applications. Notably, OMGMs played a crucial role in facilitating the controlled release of the encapsulated vaccine through a gradual enzymatic degradation mechanism, thereby significantly augmenting the therapeutic potential of subunit vaccines.

It has also been found that OMGMs exhibited remarkable motion properties under magnetic force and torque actuation, enhancing the delivery of subunit vaccines to the lymphatic system. The distinct advantages of OMGMs in subunit vaccine delivery include the utilization of OVA@MNPs with nanometer-scale dimensions, facilitating the subunit vaccine through the lymphatic intercellular space, and subsequent drainage to lymph nodes rich in APCs. Additionally, the magnetic aggregation of OVA@MNPs and the magnetic driving capabilities of OMGMs enable increased accumulation of vaccines in lymph node locations abundant in APCs. Moreover, the biodegradation of OMGMs enabled the sustained release of subunit vaccines, further enhancing antigen presentation efficiency and immune response. These combined advantages contributed to the superior drainage and targeting effects of OMGMs on subunit vaccines. For instance, in vitro experiments revealed that OMGMs significantly enhanced cross-presentation levels by more than twofold compared to free OVA (FIG. 41). Meanwhile, the secretion levels of immune response factors such as TNF-α, IL-6, and IFN-γ were increased by approximately 1.7, 1.2, and 1.3 times, respectively (FIGS. 44 to 46). In vivo experiments demonstrated that the total radiation efficiency of popliteal and inguinal lymph nodes in the OMGMs experimental group was increased by approximately 1.5 times compared to OVA alone. Meanwhile, after 36 h, the efficiency at the SLN and PLN locations in the OMGMs group was 2.3 times and 1.5 times higher than that in the OVA group, respectively.

Then, immunization experiments revealed that the proposed subunit vaccine delivery strategy achieved good anti-OVA antibody levels, indicating that OMGMs with a single booster immunization achieved antibody levels comparable to those in the case of two booster injections (FIG. 59), demonstrating the possibility of using sustained-release degradation-delivered subunit vaccines to improve immunity levels. In addition, after melanoma tumor cells were inoculated into immune mice, T-cell responses were enhanced, thereby successfully inhibiting the growth of OVA melanoma tumors (FIG. 63). These results validated the potential of the proposed vaccine delivery strategy in enhancing subunit antigen delivery efficiency and tumor immunotherapy.

This invention discloses OMGMs as a practical subunit vaccination approach, thus addressing the limitations associated with antigen delivery efficiency and paving the way for advanced immunotherapeutic strategies.

### Methods

Materials. OVA and LAP were purchased from Sigma-Aldrich. Fluorescein conjugated OVA and Alexa Fluor 647 conjugated ovalbumin were bought from Thermo Fisher Scientific. The carboxylated superparamagnetic magnetic nanoparticle solution (Fe₃O₄ MNPs-COOH, 50 nm, 25 mg mL⁻¹, suspended in double-distilled water) was customized by Chemicell GmbH. MES powder was purchased from J&K Scientific. GelMA photoresist and GelMA-FITC were bought from Engineering For Life, and all chemicals utilized were of analytical grade. Distilled water was employed for solution preparation. Table 2 lists the materials used to manufacture the OMGMs.

TABLE 2
Biodegradable OMGMs manufacturing material list

| Material | Source |
|---|---|
| Gelatin methacryloyl (GeIMA) | EFL-GM-60, Efl-tech Co., Ltd |
| GelMA-FITC | EFL-GM-GF-60, Efl-tech Co., Ltd |
| Lithium phenyl-2,4,6- | 900889, Sigma-Aldrich |
| trimethylbenzoylphosphinate (LAP) | |
| Ovalbumin | S7951, Sigma-Aldrich |
| Ovalbumin, Fluorescein Conjugate | O23020, Invitrogen |
| Ovalbumin, Alexa Fluor ™ 647 | O23020, Invitrogen |
| Conjugate | |
| Magnetic nanoparticles (MNPs) | 4115-5, fluidMAG-ARA, |
| | Chemicell GmbH |
| 2-(N-Morpholino) ethanesulfonic acid | 603282, J&K Scientific |
| monohydrate (MES) | |
| Mineral Oil | C13464990, Macklin |
| Sorbitan monooleate (Span 80) | S776246, Macklin |

Cells. DC2.4 cells were maintained in RPMI 1640 medium (Gibco, USA) supplemented with 10% FBS (Gibco, USA) and 1% penicillin-streptomycin (Invitrogen, USA) liquid at 37° C. in a 5% CO₂ incubator. B16F10-OVA cells were obtained from Hong Kong University, and the RAW264.7 macrophages were maintained in DMEM (Sigma-Aldrich, USA) supplemented with 10% FBS and 1% penicillin-streptomycin (P/S, Invitrogen). BMDCs were harvested from C57BL/6 mice following a previously established protocol.

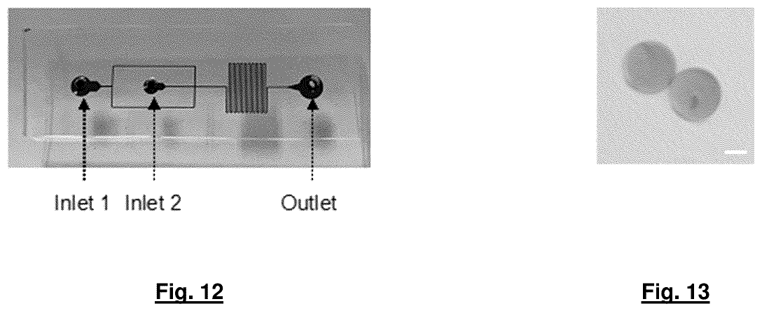

Microfluidic Device Fabrication. Droplet-generating microfluidic chips were fabricated using soft lithography. The schematic of chip fabrication and a picture of the fabricated chip is shown in FIG. 11, and a microfluidic chip fabricated for generating such droplets is shown in FIG. 12. The chip channel was coated with a surface modifier (Wei Na Biotechnology) to make it hydrophobic and stabilize droplet generation.

Synthesis and Characterization of OVA@MNPs. For the preparation of OVA@MNPs, 97.6 mg of MES powder and 50 mg of OVA powder were added to 5 mL of deionized water in two 15 mL centrifuges tubes and stirred gently at room temperature for 3-5 min. Then, OVA@MNPs conjugation was realized by incubating 20 μL of the stabilized 50 nm carboxylated MNPs (in particular Fe₃O₄ MNPs-COOH nanoparticles) solutions, 12.5 μL of 10 mg mL⁻¹ OVA, and 217.5 μL of the 0.1 mol L⁻¹ MES buffer (which is of a suitable isoelectric point) at an optimal pH overnight at room temperature with mild vortexing. The resultant product of sub-100 nm OVA@MNP subunit nanovaccines was collected by magnetic separation and washed two times with PBS. The supernatant was obtained, and the encapsulation efficiency of the OVA@MNPs was determined with the Micro BCA Protein Kit (Thermo Fisher Scientific, USA). Following relevant protocols, optimal polynomial fitting was used to determine the standard curve equation for computing the standard curve with the BCA method. Otherwise, OVA@MNPs were resuspended in PBS, stored at 4° C. for subsequent use, and protected from light (e.g., stored in a sample tube wrapped with one or more layers of tin foil). A TEM image of the OVA@MNPs was obtained with FEI Tecnai G2 F20. Nanoparticle size and zeta potential analyses were completed with Zetasizer Pro (Malvern Panalytical).

Dispersed-phase Solution Formulation. Exactly 20 mg of OVA@MNPs were added to 1 mL of PBS, and 100 mg of GelMA and 25 mg of LAP were dissolved in the PBS solution at 40° C. for 30 min. In this process, a vortex shaker was used to shake the solution several times repeatedly to accelerate the material's dissolution. In one embodiment, OVA@MNPs was added into phosphate-buffered saline. LAP was then added into the above solution at 40° C. for 30 min. GelMA was added in small amounts into the above solution several times and continuing adding after completely dissolved each time.

OMGMs Production. The dispersed-phase solution consisted of GelMA, LAP, and OVA@MNPs. The continuous-phase solution consisted of mineral oil mixed with Span 80. The flow rates of the dispersed phase and oil solutions were set to 5 and 150 μL min⁻¹, respectively, to produce OMGMs of a diameter of about 50 μm. And the production rate of OMGMs reached over 1,000 per second. Afterward, a light source of 405 nm (30 mW cm⁻²) was employed to cure the produced microspheres for 2 min. Multiple magnetic separations were performed to store the resulting microspheres in a tube. Subsequently, the OMGMs in the tube were subjected to 5-10 min of ultrasonication and multiple rounds of shaking to disperse them, aiming to minimize the occurrence of post-curing adhesion. SEM images of OMGMs and EDS results of the elemental analysis were obtained using Zeiss Sigma 300.

In one embodiment, the OMGMs in the tube were separated by (a) utilizing a pipette to suck up most of the mineral oil in the tube; (b) adding 50 μL 1H, 1H,2H,2H-Perfluoro-1-octanol (PFO) to the tube; (c) utilizing a pipette to suck out the PFO and remaining mineral oil; (d) resuspending the cured OMGMs in phosphate-buffered saline (PBS); and (e) and storing the OMGMs in a refrigerator at 4° C. PFO is here used as a microemulsion droplet demulsifier for removing the oil phase and the surfactant and separate the microspheres.

Toxicity and Biodegradation. OMGMs were seeded onto a 35 mm dish, and DC2.4 cells were introduced at a density of 5×10⁴ cells per dish. Their toxicity property was observed within 2 days using an optical microscope (Nikon). For in vitro biodegradation, OVA-FITC@MNPs were utilized to produce OMGMs. The fabricated microspheres were immersed in a PBS environment, and different volumes of the original TE (25 mg mL⁻¹) were added to configure three different concentrations of the degradation environment (5, 2.5, and 1 μg mL⁻¹). At various time points, a Zeiss fluorescence microscope was used to capture images of the three groups, and the fluorescence intensity of OMGMs was analyzed using ImageJ software. Biodegradation products were collected during the biodegradation tests. Subsequently, DC2.4 cells were seeded onto 96-well plates (Invitrogen) at a density of 1×10⁴ cells per well and cultured until reaching 40% confluence. After three washes with PBS, cells were exposed to 100 μL of a culture medium comprising varying concentrations of MNPs and biodegradation products of microspheres. The cells were cultured at 37° C. in a 5% CO₂ environment for 24 to 72 h. Following this, 20 μL of MTT solution (5 mg mL⁻¹, Sangon Biotech) was introduced to each well, and the cells were further incubated for 4 h. The incubation was then halted, and the medium was aspirated carefully. To fully dissolve the crystals, 150 μL of dimethyl sulfoxide (DMSO, Sigma-Aldrich) was added to each well, followed by gentle shaking on a low-speed shaker for 10 min. The absorbance of each well was measured at OD=490 nm using an enzyme-linked immunosorbent assay instrument (MD SpectraMAX M5e Microplate Reader).

Furthermore, the relative cell viability results were drawn using Origin software. For in vivo biodegradation, Alexa Fluor 647-OVA@MNPs and GelMA-FITC were prepared to produce OMGMs. The two kinds of fluorescence represented the two components of OMGMs that could help observe changes conveniently. Male nude mice (6-8 weeks old) received a subcutaneous injection of OMGMs containing 10 μg of OVA into the hind foot pads. The two kinds of fluorescence were visualized using an IVIS instrument (PerkinElmer) at designed time points for 72 h. The fluorescence intensity at the injection site was semi-quantified using Living 4.7.4 software. The injected area was chosen as the region of interest analysis range to obtain the total radiant efficiency, which helped the drawing of an in vivo biodegradation curve of OMGMs.

In Vitro Cellular Uptake and Cross-Presentation. DC2.4 cells and RAW264.7 macrophages were cultured at a density of 1×10⁵ cells per well in 12-well plates, with uptake experiments conducted once the cells reached 70% confluence. The cells were subsequently incubated for 2, 4, and 6 h in 1 mL of medium, containing either 5 μg of OVA-FITC or MNPs loaded with 5 μg of OVA-FITC. Following incubation, the cells were harvested, centrifuged, and subjected to three washes with PBS. Antigen uptake was then assessed using a BD BioSciences flow cytometer. Concurrently, DC2.4 cells and RAW264.7 macrophages were incubated with Alexa Fluor 647-OVA@MNPs in a confocal dish, visualizing uptake results using a confocal laser scanning microscope (LEICA TCS SP8).

DC2.4 cells were pre-exposed to specific inhibitors targeting distinct internalization pathways for a duration of 1 h. Subsequently, OVA-FITC@MNPs, containing 5 μg of OVA-FITC, were incubated for an additional hour. The inhibitors utilized included methyl-β-cyclodextrin (M-B-CD, 60 mg mL⁻¹), amiloride (5 mg mL⁻¹), dynasore (5 mg mL⁻¹), and dextran sulfate (100 mg mL⁻¹). Then, the cells were collected via centrifugation, washed thrice with PBS, and the uptake of OVA was assessed using a flow cytometer. DC2.4 cells were plated at a density of 3×10⁵ cells per 35 mm confocal culture dish. Subsequently, 2 mL of culture medium, containing 15 μg of OVA-Alexa Fluor 647@MNPs, was introduced, and the cells were incubated for 1 h. Following incubation, the OVA@MNPs were aspirated, and the cells were gently washed thrice with PBS before adding 2 mL of fresh culture medium. The cells were maintained at 37° C. in a humidified atmosphere with 5% CO₂. At designated time intervals (1 and 3 h), the cells were harvested and exposed to 2 mL of culture medium supplemented with 75 nM LysoTracker Green, followed by a 40-minute incubation at 37° C. Post-cleansing with PBS, a fresh RPMI 1640 culture medium was reinstated, and cellular observations were conducted utilizing a confocal laser scanning microscope.

Moreover, DC2.4 cells were cultured in 12-well plates at a density of 1×10⁵ cells per well. Upon reaching 70% confluence, the cells were cultured in 1 mL of medium containing either 5 μg OVA, 15 μg OVA, or OVA@MNPs loaded with 5 μg OVA for 2 h. Subsequently, DC2.4 cells were washed and further incubated for 36 h. Then, cells were harvested, subjected to three washes with PBS, stained with PE-conjugated SIINFEKL/H-2Kb anti-mouse antibody (eBioscience), and cross presentation performance was analyzed using flow cytometry.

Subsequently, BMDCs were cultured in 12-well plates at a density of 5×10⁵ cells per well and incubated with either OVA@MNPs loaded with 5 μg OVA, 5 μg OVA, or 15 μg OVA for 18 h. BMDCs supplemented with either PBS or LPS (eBioscience) served as control groups. Following incubation, cells were collected, washed three washes with PBS, and labeled with FITC-conjugated anti-mouse CD80 antibody and APC-conjugated anti-mouse CD86 antibody (eBioscience). The levels of CD80 and CD86 costimulatory molecules on BMDCs were then quantified via flow cytometry.

In Vitro Induced Immune Response. DC2.4 cells and RAW264.7 macrophages were seeded at a density of 1×10⁵ cells per well on 12-well plates until they reached 60% confluency. They were cultured in 1 mL of medium containing 5 μg free OVA, OVA@MNPs loaded with 5 μg OVA, MNPs, PBS, or LPS for 24 h. All experimental groups were replicated in three groups. Then, the cells were washed twice and centrifuged at 2,000-3,000 rpm for 20 min. The supernatant was carefully collected with a sterile tube to evaluate the secretion levels of inflammatory cytokines, including IFN-γ, IL-6, and TNF-α, by using an ELISA kit (Ruixin Biotech). Afterward, the absorbance value of each well was measured at 450 nm using the MD SpectraMAX M5e Microplate Reader, and the immune response effects characterized by IFN-γ, IL-6, and TNF-α were drawn with Origin.

Animals. C57BL/6 (female, 6-8 weeks), BALB/c (male, 6-8 weeks), and nude (female, 6-8 weeks) mice were purchased from the Laboratory Animal Research Unit (LARU), City University of Hong Kong. The mice were accommodated in a standard pathogen-free, light-cycled, temperature-regulated facility at LARU. All animal experiments were conducted strictly according to the ethical guidelines set forth by the Animal Research Ethics Sub-Committee of the City University of Hong Kong (internal reference A-0497).

In Vivo Drainage and Targeting. To explore the draining capabilities of OVA@MNPs, 10 μg of Alexa Fluor 647-OVA, and OMGMs-loaded OVA@MNPs loaded with 10 μg of Alexa Fluor 647-OVA were administered via subcutaneous injection into the hind footpad of the mice. The popliteal and sciatic lymph nodes were visualized with an IVIS instrument (PerkinElmer) at different times. The locations of varying lymph nodes were decided by relevant reference. After 36 h, the mice were humanely euthanized, and the popliteal and sciatic lymph nodes were collected for ex vivo imaging. The fluorescence intensity in the different lymph nodes was semi-quantitatively analyzed using Living Image 4.7.4 software. The injected area was chosen as the region of interest analysis range for calculating the total radiant efficiency. The radiant efficiency of the lymph nodes in the three groups were compared.

Besides, an external gradient magnetic field was added every 2 h for 10 min in a direction parallel to the mouse's hind legs. The external magnetic field was equipped with a self-constructed electromagnetic system. The other visualization operations were like those in the two other groups. With the improvement of the magnetic field, the fluorescence intensity of free OVA, OMGMs-loaded OVA@MNPs, and OMGMs-loaded OVA@MNPs with magnetic field groups were compared.

Vaccine Injection and Serum Collection. All immunizations were performed via subcutaneous injection into the hind footpad using an insulin syringe needle. Following the primary immunization on day 0, booster immunizations were administered on days 14 and 28 or solely on day 28. Blood samples (n≥3 mice per group) were drawn from the angular vein on days 14, 21, 28, and 35. Subsequently, serum was isolated through centrifugation to detect IgG antibodies. The IFN-γ and TNF-α secretion levels were detected via serum analysis using ELISA (Ruixin Biotech) on day 35.

Tumor Challenge. Seven days after the final vaccination, 5×10⁵ B16F10-OVA cells were subcutaneously injected into the right hind flank of the mice. Tumor dimensions, length, and width were measured using a Vernier caliper every 3 days. Tumor volume was computed using the formula Volume=length×width²/2.

Spleen Detection. Mice (n≥3 per group) were sacrificed on day 7 after the last vaccination, and spleens were collected and homogenized to obtain a single-cell suspension. The suspension was filtered through a 70 μm filter to remove debris. The splenocytes were treated with a red blood cell lysis buffer to eliminate red blood cells, and this buffer was utilized in subsequent experiments. For intracellular cytokine analysis of CD4⁺ T cells, splenocytes were cultured with OVA (10 mg mL⁻¹) for 10 h. Then, cells were labeled with FITC-conjugated anti-mouse CD4, APC-conjugated anti-mouse IFN-γ, and PE-conjugated anti-mouse CD3, followed by analysis via flow cytometry. Similarly, for intracellular cytokine analysis of CD8a⁺ T cells, splenocytes were cultured with OVA (10 mg mL⁻¹) for 10 h. Then, cells were labeled with FITC-conjugated anti-mouse CD8a, APC-conjugated anti-mouse IFN-γ, and PE-conjugated anti-mouse CD3 before analysis by flow cytometry.

In Vivo Biosafety. Seven days after the last vaccination, the mice were sacrificed, and their hearts, livers, spleens, lungs, and kidneys were separated and placed in 4% paraformaldehyde. The tissue was embedded in a freezing solution for cryosectioning, and the tissue sections were stained with an H&E staining kit (C0105, Biotechnology, China). Inflammation and pathological changes were then observed through a microscope. Tissue sections were prepared using a cryosectioning system (Cryostar NX70 Cryostat).

Statistical Analysis. All statistical analyses were performed with OriginPro 2023b software. All data were expressed as means±SD from at least three independent experiments. The experiments were assessed for significance by one-way ANOVA, followed by the Bonferroni method. The levels of significant differences were as follows: * P<0.05, ** P<0.01, and *** P<0.001.

1. A magnetically drivenable antigen delivery unit, including:
a magnetic nanoparticle to which a subunit antigen is loaded, and
a biodegradable material encapsulating said magnetic nanoparticle.

2. The unit of claim 1, wherein said biodegradable material is made at least partly of gelatin methacryloyl (GelMA).

3. The unit of claim 1, wherein said nanoparticle is carboxyl-modified.

4. The unit of claim 1, wherein said magnetically drivenable antigen delivery unit is in the form of a microsphere.

5. The unit of claim 1, wherein said antigen comprises ovalbumin (OVA) antigen.

6. The unit of claim 5, wherein the size of said magnetic nanoparticle loaded with said subunit antigen is below 100 nm.

7. The unit of claim 1, wherein said biodegradable material is enzymatically degradable.

8. A method of forming a magnetically drivenable antigen delivery unit, including a step (a) of forming a first solution including at least one magnetic nanoparticle to which a subunit antigen is loaded, gelatin methacryloyl (GelMA), and lithium phenyl-2,4,6-trimethylbenzoylphosphinate (LAP).

9. The method of claim 8, wherein said step (a) includes adding said magnetic nanoparticle into phosphate-buffered saline (PBS) to form a second solution, adding said LAP into said second solution to form a third solution, and adding said GelMA into said third solution to form said first solution.

10. The method of claim 8, further including a step (b) of using said first solution and a continuous phase solution to form a microsphere based on a microfluidic chip.

11. The method of claim 10, wherein said step (b) includes setting the flow rates of said first solution and said continuous phase solution on said microfluidic chip as substantially 5 μm min⁻¹ and 150 μm min⁻¹ respectively.

12. The method of claim 10, wherein said continuous phase solution comprises a mineral oil and at least one surfactant.

13. The method of claim 10, further including a step (c) of curing said microsphere with light of a preset wavelength.

14. The method of claim 13, wherein said preset wavelength is substantially 405 nm.

15. The method of claim 8, further including, prior to said step (a), a step (d) of forming said magnetic nanoparticle by loading said antigen to a carboxyl-modified magnetic nanoparticle in 2-(N-morpholino) ethanesulfonic acid (MES) buffer.

16. The method of claim 15, wherein said carboxyl-modified magnetic nanoparticle comprises an Fe₃O₄ MNPs-COOH nanoparticle.

17. The method of claim 8, wherein said antigen comprises ovalbumin (OVA) antigen.

18. A method of delivering an antigen to a subject, including injecting into said subject at least one magnetically drivenable antigen delivery unit according to claim 1.

19. The method of claim 18, further including applying a magnetic field to move said unit to or adjacent to a targeted location of said subject for subsequent release of said antigen upon degradation of said biodegradable material in said subject.

20. The method of claim 19, wherein said targeted location includes a lymph node of said subject.

21. The method of claim 10, further including a step (e) of using a microemulsion droplet demulsifier to remove the oil phase and the surfactant and separate the microspheres.

22. The method of claim 21, wherein said demulsifier is 1H, 1H, 2H,2H-Perfluoro-1-octanol (PFO).
